SEQUENCE LISTING (13 sequences)

SEQ ID NO: 1 - RNA, 16 nt, Artificial Sequence
  Synthetic RNA
cacagugaucggcauu

SEQ ID NO: 2 - RNA, 16 nt, Artificial Sequence
  Synthetic RNA
aaugugugaacguguu

SEQ ID NO: 3 - RNA, 15 nt, Artificial Sequence
  Synthetic RNA
cagugaucagcauuc

SEQ ID NO: 4 - RNA, 16 nt, Artificial Sequence
  Synthetic RNA
agaaugugaucacugu

SEQ ID NO: 5 - RNA, 16 nt, Artificial Sequence
  Synthetic RNA
aggccugcagaucuuu

SEQ ID NO: 6 - RNA, 16 nt, Artificial Sequence
  Synthetic RNA
aauugagcaacaggcc

SEQ ID NO: 7 - RNA, 16 nt, Artificial Sequence
  Synthetic RNA
aguccaauuuccagca

SEQ ID NO: 8 - RNA, 16 nt, Artificial Sequence
  Synthetic RNA
uuuugcuauuggccuu

SEQ ID NO: 9 - RNA, 16 nt, Artificial Sequence
  Synthetic RNA
gccaauuuccagcaaa

SEQ ID NO: 10 - RNA, 16 nt, Artificial Sequence
  Synthetic RNA
cacagugaucggcauu

SEQ ID NO: 11 - RNA, 16 nt, Artificial Sequence
  Synthetic RNA
guauccuaauggugua

SEQ ID NO: 12 - RNA, 16 nt, Artificial Sequence
  Synthetic RNA
  modified_base (1)..(1): gm
  modified_base (2)..(2): um
  modified_base (3)..(3): am
  modified_base (12)..(12): gm
  modified_base (16)..(16): am
guauccuaauggugua

SEQ ID NO: 13 - RNA, 16 nt, Artificial Sequence
  Synthetic RNA
  modified_base (1)..(1): gm
  modified_base (2)..(2): um
  modified_base (3)..(3): am
  modified_base (4)..(4): um
  modified_base (5)..(5): cm
  modified_base (6)..(6): cm
  modified_base (7)..(7): um
  modified_base (8)..(8): am
  modified_base (9)..(9): am
  modified_base (10)..(10): um
  modified_base (11)..(11): gm
  modified_base (12)..(12): gm
  modified_base (13)..(13): um
  modified_base (14)..(14): gm
  modified_base (15)..(15): um
  modified_base (16)..(16): am
guauccuaauggugua

(19) United States
(12) Patent Application Publication
(10) Pub. No.: US 2024/0254149 A1
(43) Pub. Date: Aug. 1, 2024

(54) Chemical Phosphorylation Reagents, Preparation, and Their Uses

(71) Applicant: OliX US, Inc., San Diego, CA (US)

(72) Inventors: Dongwon Shin, San Diego, CA (US); Namho Kim, San Diego, CA (US); Raymond Emehiser, Santee (CA)

(21) Appl. No.: 18/563,135

(22) PCT Filed: May 20, 2022
(86) PCT No.: PCT/US2022/030253

§ 371 Date: Nov. 21, 2023

Related U.S. Application Data
(60) Provisional application No. 63/328,190, filed on Apr. 6, 2022.
(60) Provisional application No. 63/191,721, filed on May 21, 2021.

(51) Int. Cl. C07F 9/24; B01D 15/32; B01D 15/36; C07H 1/06; C07H 21/02

(52) U.S. Cl. CPC C07F 9/2408; B01D 15/325; B01D 15/361; C07H 1/06; C07H 21/02

(57) ABSTRACT

Described herein are chemical phosphorylating reagents, their synthesis, and uses thereof.

### SEQUENCE LISTING

This application contains a sequence listing having the filename 0817444-00013_ST25.txt, which is 3,956 bytes in size, and was created on May 18, 2022. The entire content of this sequence listing is incorporated herein by reference.

### RELATED APPLICATIONS

This application is a national stage entry under 35 USC § 371 of International Patent Application No. PCT/US2022/030253, filed May 20, 2022, which claims priority of U.S. Provisional Patent Application Nos. 63/191,721, filed May 21, 2021, and 63/328,190, filed Apr. 6, 2022, the entire contents of which are incorporated herein by reference.

### FIELD

Described herein are chemical phosphorylating reagents, their synthesis, their synthetic intermediates, and uses thereof.

### BACKGROUND

Preparation of oligomers, including oligonucleotides, carbohydrates, peptides, or the like, may be performed via iterations of synthetic cycles. For example, deoxyribonucleic acid (DNA) synthesis may comprise a first monomer bound to a solid support on which an oligomer of DNA is prepared by cycling through steps including deblocking the first monomer, and coupling of a second monomer to the first monomer. Optional steps include capping of uncoupled first monomers, and oxidation. A cycle of these steps may generate the desired length and sequence of molecule, which cycle is then ended upon final processing of the oligomer including a final deprotection sequence and deblocking of, typically, a trityl moiety, and purification. Similar cycles are utilized for synthesizing peptides, carbohydrates, or other molecules amenable to preparation by iterative synthesis cycling. Many commercial entities provide services to prepare molecules in this way, including Glen Research, Integrated DNA Technologies, Panagene, GlycoUniverse, CSBio, as well as many others. A variety of benchtop machines are available for researchers to build their own molecules, including Kilobaser, Biolytic's Dr. Oligo series, Biolytic's ABI series, the MerMade series, the Expedite series, the Glyconeer, the Biotage series, and many other synthesizers. However, the need for chemical phosphorylating reagents remains unsatisfied due in part to problems arising from the particular sequences required for deprotection and purification to obtain the desired phosphorylated molecule.

Phosphorylated molecules (e.g., oligomers, e.g., oligonucleotides possessing a 5′-phosphate group) have been pursued for a long time due to their multiple purposes. For example, phosphorylated oligonucleotides provide valuable tools for gene construction, cloning, mutagenesis, ligation chain reaction, and many other biological applications. Enzymatically, T4 polynucleotide kinase is well known to catalyze the transfer of γ-phosphate from adenosine 5′-triphopshate (ATP) to the 5′-terminus of oligonucleotides or to mononucleotides bearing a 5′-hydroxymethyl group. There have been chemical 5′-phosphorylation methods developed to overcome limitations of enzymatic synthesis. Some methods include the preparation of modified nucleoside-based building blocks containing 5′-phosphate analogs to be attached at the last step of oligomer (e.g., oligonucleotide) synthesis. Other methods utilize non-nucleosidic building blocks, namely, the chemical phosphorylation reagents. Chemical phosphorylation reagents have been used for the preparation of phosphorylated oligonucleotides and a variety of approaches have been elaborated to introduce phosphate compatible analogues such phosphotriester, H-phosphonate, methyl phosphonamidite, or phosphoramidite (Glen Research Corporation, U.S. Pat. No. 5,959,090).

Many chemical reagents have been developed to monitor the efficiency of chemical coupling reaction for phosphate analog to oligonucleotides. One phosphorylation reagent utilizes the orthogonal protection of 5′-phosphate containing a 2-(tritylthio)ethyl functional moiety. 5′-phosphate of an oligonucleotide was obtained by cleavage of the tritylthio bond under aqueous silver nitrate or iodine followed by addition of dithiothreitol at pH 8.5 affording ethylene sulfide. However, its usage was only recognized as an introduction of modified 5′-phosphate functional group.

A building block derived from (4,4′-dimethoxytrityloxyethyl) hydroxyethyl sulfone was first introduced to monitor the coupling reaction colorimetrically, easily and accurately (Chiron corporation, U.S. Pat. No. 5,257,760; Tetrahedron Lett., 1986, 27, 4705). This chemical phosphorylation reagent (namely, CPR-I) is used for the synthesis of 5′-phosphorylated oligonucleotides. This phosphorylation reagent contains a 4,4′-dimethoxytrityl group that is cleavable (deblocked) with acid and colorimetrically detectable upon release (that is, conventional dimethoxytrityl (DMT) assay). Ammonolytic deprotection of chemically phosphorylated oligonucleotide results in β-elimination of the O-phosphorylated hydroxyethyl sulfone fragment affording an oligonucleotide possessing a 5′-phosphate group. However, this means that the 4,4′-diemthoxytrityl group is lost on release of 5′-phosphate group so the oligonucleotide purification utilizing the trityl specific isolation (namely, DMT-ON purification) is not possible. Furthermore, the deprotection conditions affording the β-elimination reaction to completion often demonstrate the decomposition of 5′-phosphorylated oligonucleotides due to their harsh conditions including the incubation with ammonium hydroxide for 18 hours at about room temperature or 4 hours at about 55-65° C. In addition, it is not compatible with the standard oligonucleotide deprotection condition utilizing AMA (1:1 ammonium hydroxide and methylamine solution), which cleaves base-labile protecting groups utilized during the synthesis cycle.

Chemical phosphorylation reagent II (CPR II) is another chemical phosphorylation reagent for the synthesis of 5′-phosphorylated oligonucleotides (Tetrahedron, 1995, 51, 9375). While CPR-I was incompatible with DMT-ON purification, CPR II allowed both DMT-on purification and DMT-off purification. 5′-phosphorylated oligonucleotides are obtained by two step reactions including DMT-deprotection and elimination. After deprotection of DMT under mild acidic condition, the elimination is initiated by deprotonation of terminal hydroxyl group to generate formaldehyde and subsequently to afford the 5′-phosphate. Solid chemical phosphorylation reagent II (Solid CPR II) was developed to overcome the drawback of CPR II as liquid. Solid CPR II is also compatible to both of DMT-on and DMT-off purification, and its transformation to 5′-phosphate follows the same condition (J Biol. Chem., 2000, 275, 22355). However, both CPR II and Solid CPR II are not compatible with synthetic preparation of wildtype 5′-phosphorylated RNA oligonucleotide due to the degradation by intramolecular 2′,3′-cyclicphosphate formation under strong basic condition (e.g., standard deprotection using AMA).

Currently, oligonucleotide purification is commonly selected from reverse phase (RP) column chromatography and/or anion exchange (AEX) column chromatograph. Additionally, oligonucleotide is also purified by oligonucleotide purification cartridge (OPC) utilizing the hydrophobicity and easy deprotection of trityl group. Column chromatography is widely applied for the purification of oligonucleotide regardless the phosphorylation or any conjugation linker attached on oligonucleotide. In spite of its high purification efficiency over 95% purity, these column chromatographic tools are limited due to time consuming process including purification, concentration, and desalting. Oligonucleotide purification by OPC is less efficient in terms of purity. But it gives a faster and easier way for massive purification of oligonucleotides. Recent development for OPC has been leading the higher purity of oligonucleotide in a couple of hours. Specifically, the preparative reverse phase separation of 5′-phosphorylated oligonucleotide is often inefficient from the corresponding non-phosphorylated oligonucleotide. Although the anion exchange separation is more efficient for 5′-phosphorylated oligonucleotides, it is also restricted by the length of oligonucleotide fragments to be isolated. Eventually, 5′-phosphorylated DNA oligonucleotides and fully modified 5′-phosphorylated RNA oligonucleotides can be purified by OPC utilizing the DMT-ON purification. However, it still needs a subsequent time-consuming purification period for the 5′-phosphorylated RNA oligonucleotides by RP or AEX column chromatography.

Thus, provided herein are new chemical phosphorylation reagents, their use in the synthesis and phosphorylation of molecules, including oligomers such as DNA or RNA, and methods of their use in purification of such phosphorylated molecules. For example, the chemical phosphorylation reagents herein can follow the general solid phase synthesis cycle for 5′-phosphorylation, DMT-on OPC purification, and are compatible with transformation to 5′-phosphate under the mild condition to retain the structure of 5′-phosphorylated RNA oligonucleotides.

### SUMMARY

Described herein are compounds, having the formula:

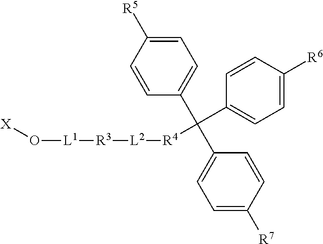

or a salt thereof.

Also provided herein are methods of preparing the compounds, methods of phosphorylating a molecule with the compounds, and methods of purifying such phosphorylated molecules.

### BRIEF DESCRIPTION OF THE DRAWINGS

FIG. 1 shows a synthetic scheme useful for preparing the compounds described herein, and their synthetic intermediates. Moieties of the chemical formulae are as provided herein.

FIG. 2 shows a synthetic scheme useful for preparing compound 5, and its synthetic intermediates.

FIG. 3 shows another synthetic scheme useful for preparing the compounds described herein, and their synthetic intermediates. Moieties of the chemical formulae are as provided herein.

FIG. 4 shows a scheme for phosphorylating an oligonucleotide using a compound provided herein.

FIG. 5 shows a scheme for purifying 5′-phosphorylated oligonucleotide by OPC.

FIG. 6 shows HPLC analysis of a wildtype phosphorylated RNA oligonucleotide prepared and processed according to commercial procedures with commercially available Solid CPR II.

FIG. 7 shows the structures of certain moieties referred to herein by an abbreviated phrase identifier.

FIG. 8 shows a synthetic scheme useful for preparing compound 10, and its synthetic intermediates.

FIG. 9 shows a scheme for phosphorylating an oligonucleotide using a compound provided herein.

FIG. 10 shows the structure (and naming terminology) of non-limiting examples of nucleotide monomers, that may be used to prepare oligonucleotides referred to herein.

FIG. 11 shows a scheme for purifying 5′-phosphorylated oligonucleotide by OPC.

FIG. 12 shows LC/MS traces for 10 wildtype strands (samples A-J) of 5′-phosphorylated RNA oligonucleotides with DMTr thioethyl tether in 100 nmol scale purified by OPC purification corresponding to Example 9a.

FIG. 13 shows chromatographic traces for 3 strands (samples A-F; crude and purified) of 5′-phosphorylated RNA oligonucleotides with DMTr thioethyl tether in 10 μmol scale purified by OPC purification corresponding to Example 9b (Peak Index: 1. 5′-[DMTr-TE-Phos]-RNA oligonucleotide-3′; 2. 5′-[Phos]-RNA oligonucleotide-3′; 3. Failure sequences from oligonucleotide synthesis).

### DETAILED DESCRIPTION

As described throughout, including in the Examples below, it has been found that compounds described herein are useful in chemically phosphorylating other molecules. It has also been found that the compounds described herein are useful in purifying other molecules after having been phosphorylated with the compounds provided herein. The chemical phosphorylating compounds provided herein are stable to the synthetic methods required to iterate through synthesis cycles, deprotect and purify molecules, including synthetically prepared oligomers. For example, phosphorylated molecules prepared with phosphorylating reagents described herein may be obtained by the simpler process of OPC purification in purity when compared to time consuming and labor intensive RP-HPLC or AEX purification techniques (e.g., Examples 11-14).

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The terms “composition” refers to a mixture of at least one compound described herein with a carrier.

The term “carrier” means an acceptable material, including a liquid filler, solid filler, stabilizer, dispersing agent, suspending agent, diluent, excipient, thickening agent, solvent, or encapsulating material, involved in carrying or transporting at least one compound described herein within or to a desired location such that the compound may perform its intended function. A given carrier must be “acceptable” in the sense of being compatible with the other ingredients of a particular composition, including the compounds described herein, and not injurious to the compounds therein.

The term “oligomer” refers to a polymer made up of a few repeating parts, e.g., monomers, which may or may not be identical, and may include similar chemical features.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability.

Reference made to a patent document or other publication in this specification serves as an incorporation herein by reference of the entire content of such document or publication.

Embodiments of this disclosure are illustrative. Accordingly, the present disclosure is not limited to that precisely as shown and described.

### Compounds

Chemical phosphorylation compounds are described herein. Also described herein are synthetic intermediate compounds useful in the preparation of the chemical phosphorylation compounds described herein. The phrases “chemical phosphorylation compound” or “chemical phosphorylation reagent” are meant to be interchangeable.

In some embodiments, described herein are compounds, having the formula:

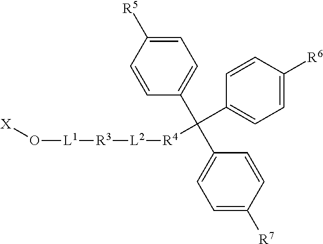

or a salt thereof,
wherein,
X is H,

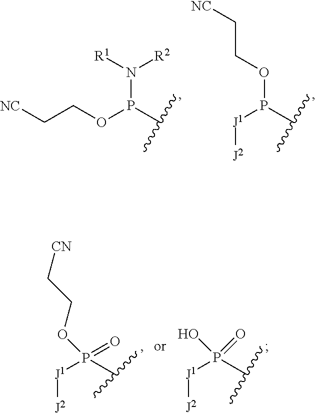

J¹ is O, NH, or S;
J² is a macromolecule;
R¹ is C₁₋₆ alkyl;
R² is C₁₋₆ alkyl;
L¹ is C₁₋₆ alkylene, C₁₋₆ alkenylene, or C₁₋₆ alkynylene;
R³ is a bond, O, N(H), N(CH₃), S, C₂₋₆ alkylene, C₂₋₆ alkenylene, C₂₋₆ alkynylene, SS, O—N═C, C═N—O, C(O)N(H), N(H)C(O), OC(O)O, OC(O)N(H), N(H)C(O)O, N(H)C(O)N(H), OC(O)N(C₁₋₆ alkyl), N(C₁₋₆ alkyl)C(O)O, N(C₁₋₆ alkyl)C(O)N(C₁₋₆ alkyl), C(H)═N, or N═C(H);
L² is C₁₋₆ alkylene, C₁₋₆ alkenylene, or C₁₋₆ alkynylene;
R⁴ is 0 or S;
R⁵ is H or an electron donating moiety;
R⁶ is H or an electron donating moiety; and
R⁷ is H or an electron donating moiety;
wherein the compound is not 2-((2-(bis(4-methoxyphenyl)(phenyl)methoxy)ethyl)disulfaneyl)ethyl (2-cyanoethyl) diisopropylphosphoramidite or 2-((2-(bis(4-methoxyphenyl)(phenyl)methoxy)ethyl)disulfaneyl)ethan-1-ol.

In some embodiments, described herein are compounds, having the formula:

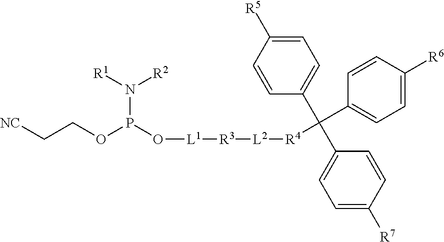

wherein,
R¹ is C₁₋₆ alkyl;
R² is C₁₋₆ alkyl;
L¹ is C₁₋₆ alkylene, C₁₋₆ alkenylene, or C₁₋₆ alkynylene;
R³ is a bond, O, N(H), N(CH₃), S, CₙH₂ₙ₊₂, CₙH₂ₙ, CₙH₂ₙ₋₂, where n is more than 2, SS, C(CₘH₂ₘ₊₂)₂, where m is more than 1, O—N═C, C═N—O, C(O)N(H), N(H)C(O), OC(O)O, OC(O)N(H), N(H)C(O)O, N(H)C(O)N(H), OC(O)N(CₓH₂ₓ₊₂), N(CₓH₂ₓ₊₂)C(O)O, N(CₓH₂ₓ₊₂)C(O)N(C_{y}H_{2y+2}), where x and y are each, independently, more than 1, C═N, or N═C;
L² is C₁₋₆ alkylene, C₁₋₆ alkenylene, or C₁₋₆ alkynylene;
R⁴ is O or S;
R⁵ is H or an electron donating moiety;
R⁶ is H or an electron donating moiety; and
R⁷ is H or an electron donating moiety. In some embodiments, the compound is not 2-((2-(bis(4-methoxyphenyl)(phenyl)methoxy)ethyl)disulfaneyl)ethyl (2-cyanoethyl) diisopropylphosphoramidite. In some embodiments, R³ is a bond, O, N(H), N(CH₃), S, C₂₋₆ alkylene, C₂₋₆ alkenylene, C₂₋₆ alkynylene, SS, O—N═C, C═N—O, C(O)N(H), N(H)C(O), OC(O)O, OC(O)N(H), N(H)C(O)O, N(H)C(O)N(H), OC(O)N(C₁₋₆ alkyl), N(C₁₋₆ alkyl)C(O)O, N(C₁₋₆ alkyl)C(O)N(C₁₋₆ alkyl), C(H)═N, or N═C(H). In some embodiments, if R³ is SS (disulfide), R⁴ is O, and L¹ and L² are independently C₁₋₆ alkylene, then L¹ and L² are different. In some embodiments, when R³ is SS (disulfide) and R⁴ is O, L¹ and L² are different. In some embodiments, when R⁴ is O, one or more of the following applies: 1) L¹ is methylene, a propylene, a butylene, a pentylene, a hexylene, C₁₋₆ alkenylene, or C₁₋₆ alkynylene; 2) R³ is a bond, O, N(H), N(CH₃), S, C₂₋₆ alkylene, C₂₋₆ alkenylene, C₂₋₆ alkynylene, O—N═C, C═N—O, C(O)N(H), N(H)C(O), OC(O)O, OC(O)N(H), N(H)C(O)O, N(H)C(O)N(H), OC(O)N(C₁₋₆ alkyl), N(C₁₋₆ alkyl)C(O)O, N(C₁₋₆ alkyl)C(O)N(C₁₋₆ alkyl), C(H)═N, or N═C(H); or 3) L² is methylene, a propylene, a butylene, a pentylene, a hexylene, C₁₋₆ alkenylene, or C₁₋₆ alkynylene. In some embodiments, the electron donating moiety is an alkoxy moiety, e.g., O—(C₁₋₆ alkyl). In some embodiments, each occurrence of C₁₋₆ alkyl, whether alone or in combination with another group, refers, independently, to methyl, ethyl, or a propyl (e.g., n- or i-propyl). In some embodiments: R¹ and R² are isopropyl; L¹ and L² are ethylene; R³ is O, NH, NCH₃, S, O—N═CH, CH═N—O, C(O)NH, NHC(O), OC(O)O, OC(O)NH, NHC(O)O, or NHC(O)NH; and R⁵, R⁶, and R⁷ are each, independently, H or OCH₃. In some embodiments, L¹-R³-L² is CH₂CH₂. In some embodiments, L¹ and L² are each CH₂. In some embodiments, L² is CH₂CH₂ and R³ is other than a bond or SS.

In some embodiments, the compounds have a formula according to:

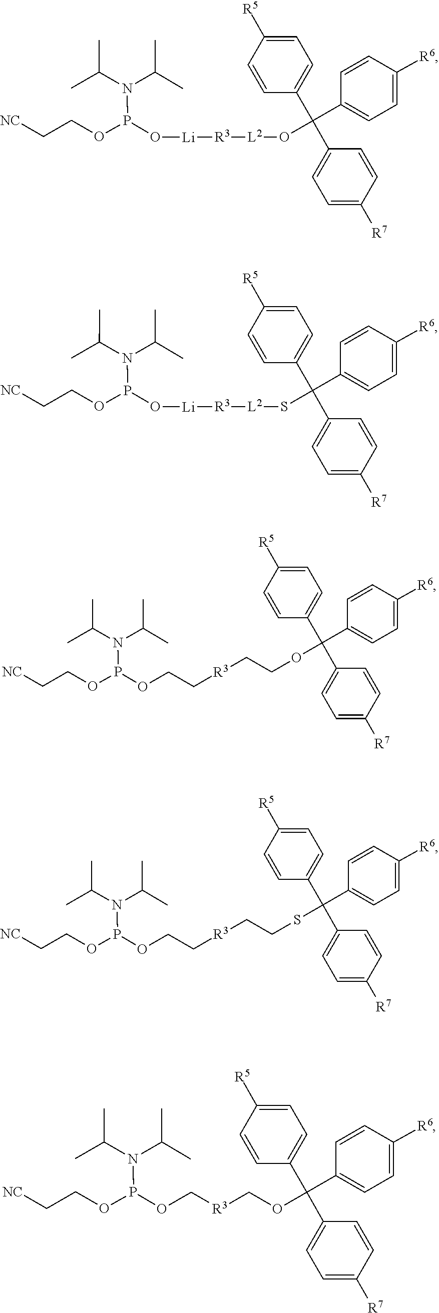

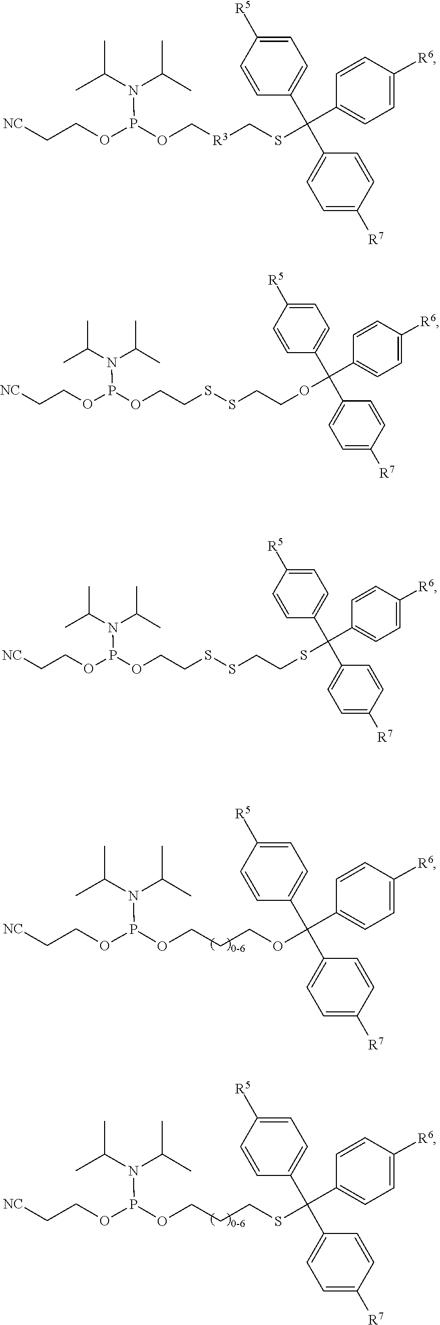

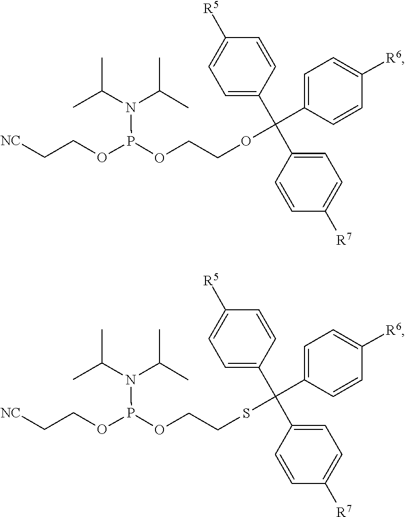

wherein the compound is not 2-((2-(bis(4-methoxyphenyl)(phenyl)methoxy)ethyl)disulfaneyl)ethyl (2-cyanoethyl) diisopropylphosphoramidite. In some embodiments of the formulae herein, R⁵, R⁶, and R⁷ are H. In some embodiments, R⁵ and R⁶ are H and R⁷ is OCH₃. In some embodiments, R⁵ is H and R⁶ and R⁷ are OCH₃. In some embodiments, R⁵, R⁶, and R⁷ are OCH₃.

In some embodiments of the formulae herein, 1) R³ is SS (disulfide), 2) R¹ and R² are isopropyl, 3) L¹ and L² are CH₂CH₂, 4) R⁵, R⁶, and R⁷ are each, independently, H or OCH₃, or 5) a combination thereof.

In some embodiments, the compound of the formulae provided herein is selected from a compound of Table 1.

TABLE 1
Selected compounds provided herein.
Compound

| | |
|---|---|
| 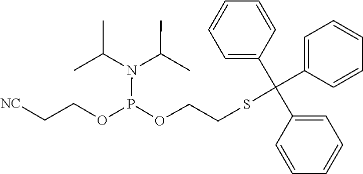 [SMILES: CC(C)N(C(C)C)P(OCCC#N)OCCSC(c1ccccc1)(c1ccccc1)c1ccccc1] | 1 |
| 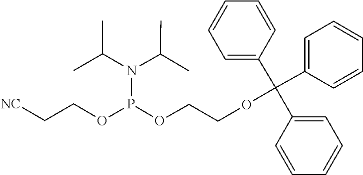 [SMILES: CC(C)N(C(C)C)P(OCCC#N)OCCOC(c1ccccc1)(c1ccccc1)c1ccccc1] | 2 |
| 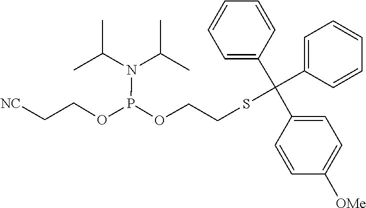 [SMILES: COc1ccc(C(SCCOP(OCCC#N)N(C(C)C)C(C)C)(c2ccccc2)c2ccccc2)cc1] | 3 |
| 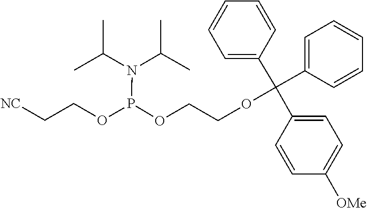 [SMILES: COc1ccc(C(OCCOP(OCCC#N)N(C(C)C)C(C)C)(c2ccccc2)c2ccccc2)cc1] | 4 |
| 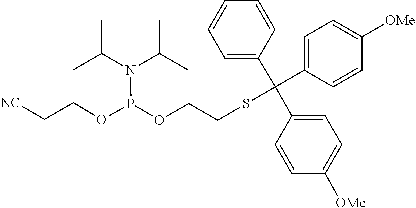 [SMILES: COc1ccc(C(SCCOP(OCCC#N)N(C(C)C)C(C)C)(c2ccccc2)c2ccc(OC)cc2)cc1] | 5 |
| 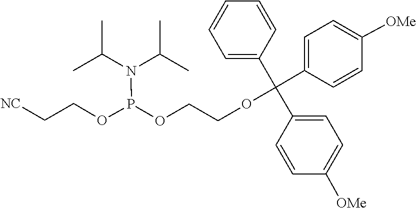 [SMILES: COc1ccc(C(OCCOP(OCCC#N)N(C(C)C)C(C)C)(c2ccccc2)c2ccc(OC)cc2)cc1] | 6 |
| 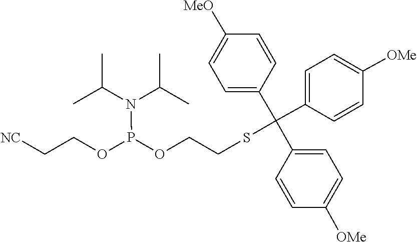 [SMILES: COc1ccc(C(SCCOP(OCCC#N)N(C(C)C)C(C)C)(c2ccc(OC)cc2)c2ccc(OC)cc2)cc1] | 7 |
| 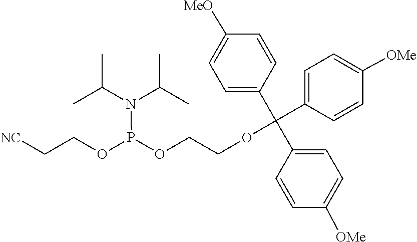 [SMILES: COc1ccc(C(OCCOP(OCCC#N)N(C(C)C)C(C)C)(c2ccc(OC)cc2)c2ccc(OC)cc2)cc1] | 8 |
| 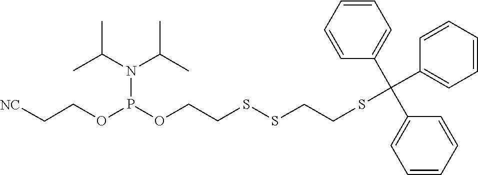 [SMILES: CC(C)N(C(C)C)P(OCCC#N)OCCSSCCSC(c1ccccc1)(c1ccccc1)c1ccccc1] | 9 |
| 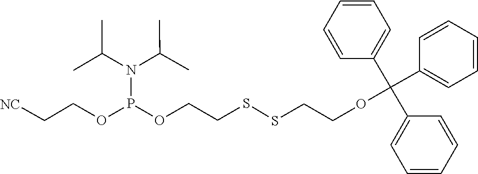 [SMILES: CC(C)N(C(C)C)P(OCCC#N)OCCSSCCOC(c1ccccc1)(c1ccccc1)c1ccccc1] | 10 |
| 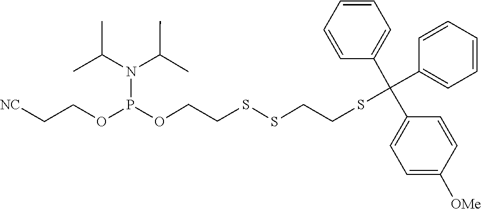 [SMILES: COc1ccc(C(SCCSSCCOP(OCCC#N)N(C(C)C)C(C)C)(c2ccccc2)c2ccccc2)cc1] | 11 |
| 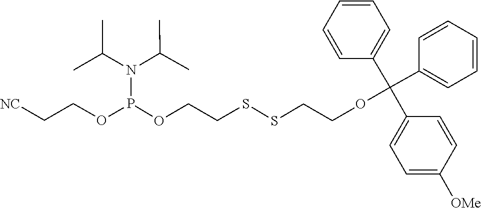 [SMILES: COc1ccc(C(OCCSSCCOP(OCCC#N)N(C(C)C)C(C)C)(c2ccccc2)c2ccccc2)cc1] | 12 |
| 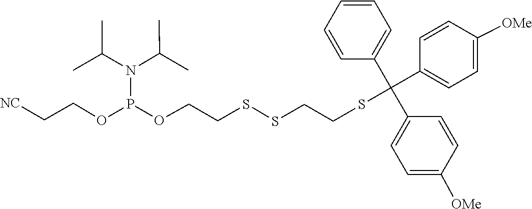 [SMILES: COc1ccc(C(SCCSSCCOP(OCCC#N)N(C(C)C)C(C)C)(c2ccccc2)c2ccc(OC)cc2)cc1] | 13 |
| 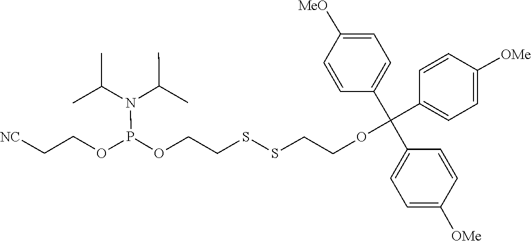 [SMILES: COc1ccc(C(OCCSSCCOP(OCCC#N)N(C(C)C)C(C)C)(c2ccc(OC)cc2)c2ccc(OC)cc2)cc1] | 14 |
| 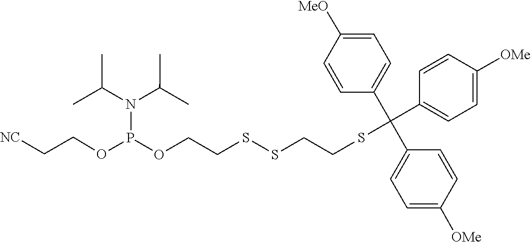 [SMILES: COc1ccc(C(SCCSSCCOP(OCCC#N)N(C(C)C)C(C)C)(c2ccc(OC)cc2)c2ccc(OC)cc2)cc1] | 15 |
| 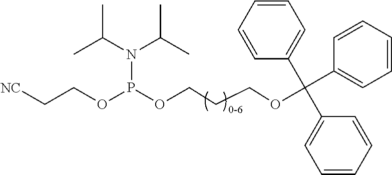 | 16 |
| 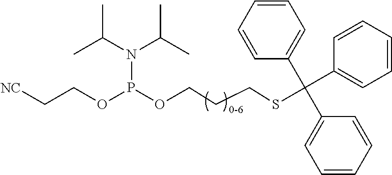 | 17 |
| 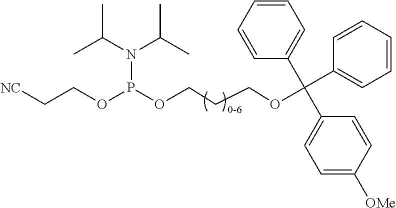 | 18 |
| 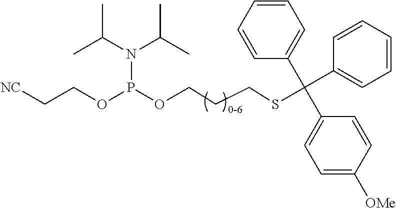 | 19 |
| 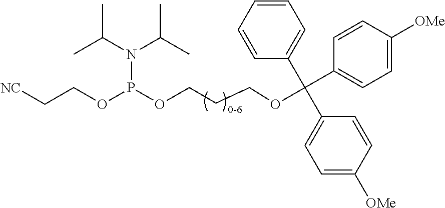 | 20 |
| 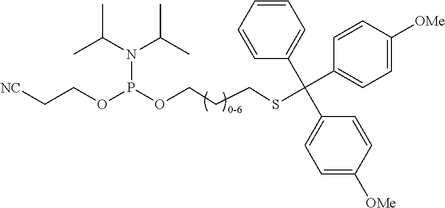 | 21 |
| 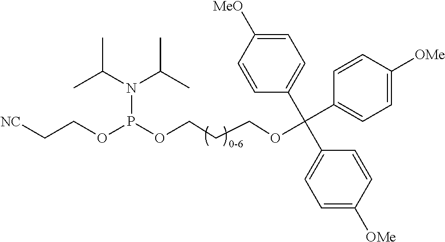 | 22 |
| 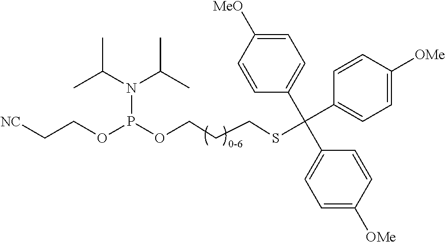 | 23 |

Compounds described herein also include isotopically-labeled compounds wherein one or more atoms is replaced by an atom having the same atomic number, but an atomic mass or mass number different from the atomic mass or mass number predominantly found in nature. Examples of isotopes suitable for inclusion in the compounds described herein include and are not limited to ²H, ³H, ¹¹C, ¹³C, ¹⁴C, ¹³N, ¹⁵N, ¹⁵O, ¹⁷O, ¹⁸O, ³²P, and ³⁵S. In some embodiments, isotopically-labeled compounds are useful in drug or substrate tissue distribution studies, afford greater metabolic stability, or are useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In some embodiments, the compounds described herein are labeled by other means, including, but not limited to, the use of chromophores or fluorescent moieties, bioluminescent labels, or chemiluminescent labels.

### Compositions

Also described herein are compositions comprising the compounds described herein. The compositions may include one or more acceptable carriers. In some embodiments, the carrier is a solvent or an inert stabilizer, or both. In some embodiments, the inert stabilizer provides a dehydrating effect to the composition, which may enable a longer shelf life stability of the compounds for storing the composition.

Articles of manufacture are described, which comprise the compounds or compositions provided herein. The articles of manufacture may include forms of the compounds or compositions suitable for administration or storage. In some embodiments, the article of manufacture may take the form of, and also may be administered as, an ingestible, an inhalable, an injectable, or a depositable article. In some embodiments, the form may be an orally ingestible article, or a suppository. In some embodiments, the form is a solid form, such as a powder or lyophilized form.

The present compounds and associated materials can be finished as a commercial product by the usual steps performed in the present field, for example by appropriate sterilization and packaging steps while maintaining the compound under inert or anhydrous conditions. The material according to the present disclosure can be finally sterile-wrapped so as to retain sterility until use and packaged (e.g., by the addition of specific product information leaflets) into suitable containers (boxes, etc.).

According to further embodiments, the present compounds can also be provided in kit form combined with other components necessary for use of the material in phosphorylating molecules, optionally including one or more of containers, reagents, or instructions.

The compounds or compositions provided herein may be prepared and placed in a container for storage at ambient or elevated temperature. When the compound or composition is stored in a polyolefin plastic container as compared to a polyvinyl chloride plastic container, discoloration of the compound or composition may be reduced, whether dissolved or suspended in a liquid composition (e.g., an anhydrous organic liquid solution), or as a syrup or solid. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce exposure of the container's contents to infrared light, or a second component with such a capacity. Some containers also include the capacity to reduce exposure of the container's contents to water, or a second component with such capacity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper, cardboard, paperboard, metallic film, or foil, or a combination thereof, container to further reduce exposure of the container's contents to UV, visible, or infrared light. Compounds and compositions benefiting from reduced discoloration, decomposition, or both during storage, include liquid or syrup solutions that include a compound or composition thereof provided herein. The compounds or compositions provided herein may need storage lasting up to, or longer than, three months; in some cases up to, or longer than one year. The containers may be in any form suitable to contain the contents; for example, a bag, a bottle, or a box, or a combination thereof.

In some embodiments, provided herein are packaged compounds, packaged compositions, or packaged pharmaceutical compositions, e.g., kits, comprising a container housing an effective amount of a compound described herein, and instructions for using the compound in accordance with one or more of the methods or uses provided herein.

### Synthesis

The compounds described herein are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as described herein are modified by the use of appropriate reagents and conditions, for the introduction of the various moieties found in the formula as provided herein. Compounds described herein are synthesized using any suitable procedures starting from compounds that are available from commercial sources, or are prepared using procedures described herein.

In some embodiments, reactive functional groups, such as hydroxyl, amino, imino, thio, or carboxy groups, are protected. Protecting groups are used to block some or all of the reactive moieties and prevent such groups from participating in chemical reactions until the protective group is removed. In some embodiments, protective groups are removed by acid, base, reducing conditions (for example, by hydrogenolysis), or oxidative conditions.

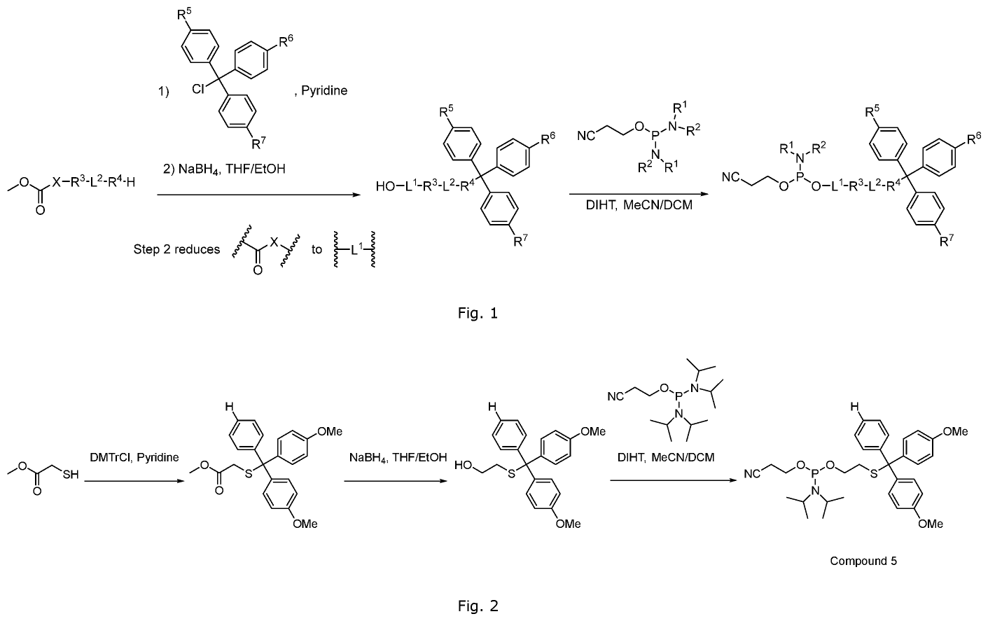

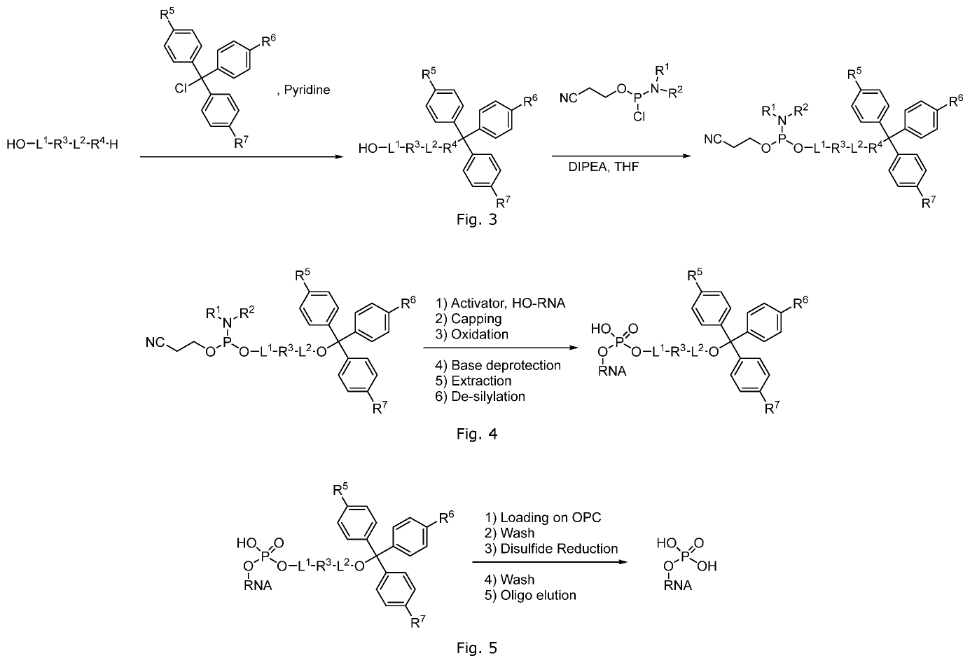

In some embodiments, the compounds described herein may be prepared by a method of synthesis that comprises any one of the synthetic steps or schemes shown in FIG. 1, FIG. 2, FIG. 3, FIG. 4, FIG. 5, or FIG. 8, or a combination thereof.

In some embodiments, the phosphorylated molecules provided herein may be prepared or purified (e.g., by OPC purification as provided herein), or both, on any suitable scale, including about 100 nmol, about 1 μmol, about 10 μmol, about 20 μmol, or more than 20 μmol scale, e.g., about 0.1 mmol scale or more, or any range therebetween, e.g., about 100 nmol to about 0.1 mmol.

In some embodiments, the phosphorylated molecules provided herein may be present in a composition at a concentration of about 1 fM or more, e.g., about 1 μM, about 10 μM, about 100 μM, about 1 mM, about 10 mM, or more, and optionally at a purity of about 70% or more, about 85% or more, e.g., about 90%, about 95%, about 98%, about 99%, or about 99.5% or more, about 99.9% or more, or any range therebetween, e.g. about 1 fM to about 10 mM at about 70% to about 99.9% purity.

### Methods

In some embodiments, provided herein are methods of phosphorylating a molecule, comprising contacting the molecule with a compound provided herein, which compound includes a phosphorous atom, such that the phosphorous atom forms a covalent bond with the molecule. In some embodiments, the yield of the phosphorylation reaction is at least 30%, at least 40%, at least 50%, at least 60%, at least 70%, at least 80%, at least 90%, at least 95%, at least 98%, or at least 99%. In some embodiments, the molecule comprises an oligomer. In some embodiments, the molecule comprises a nucleic acid, a nucleic acid analog, a peptide, or an oligosaccharide.

In some embodiments, provided herein are methods for synthesis and purification of oligonucleotides possessing 5′-phosphate groups. In some embodiments, the methods of synthesis include phosphoramidite-based building blocks.

In some embodiments of these methods, the molecules or oligomers that may be phosphorylated by the compounds provided herein, include oligonucleotides, carbohydrates, peptides, analogs thereof, or the like. In some embodiments, an oligomer may include nucleic acid monomers, nucleic acid mimic monomers (e.g., monomers including a nonstandard sugar moiety, a nonstandard backbone, a modified nucleobase, a moiety that may be capable of orthogonal pairing analogous to a nucleobase, or a moiety that occupies the structural/steric space of a nucleobase but has little to no capacity for orthogonal pairing, or a combination thereof), amino acid monomers, amino acid mimic monomers (e.g., monomers including a non-standard side chain, non-standard stereochemistry, non-standard backbone, or a combination thereof), saccharide monomers or analogs thereof, or mixtures thereof.

In some embodiments, an oligonucleotide that is phosphorylated by a compound herein is phosphorylated at the oligonucleotide's 5′- or 3′-end.

In some embodiments, a molecule phosphorylated as described herein is phosphorylated at a hydroxyl, thiol, or amine moiety on the molecule.

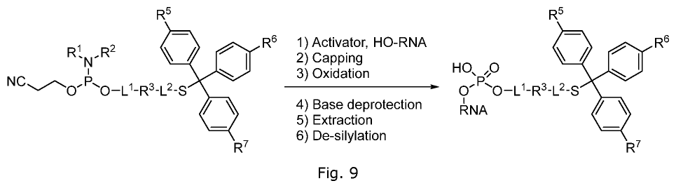

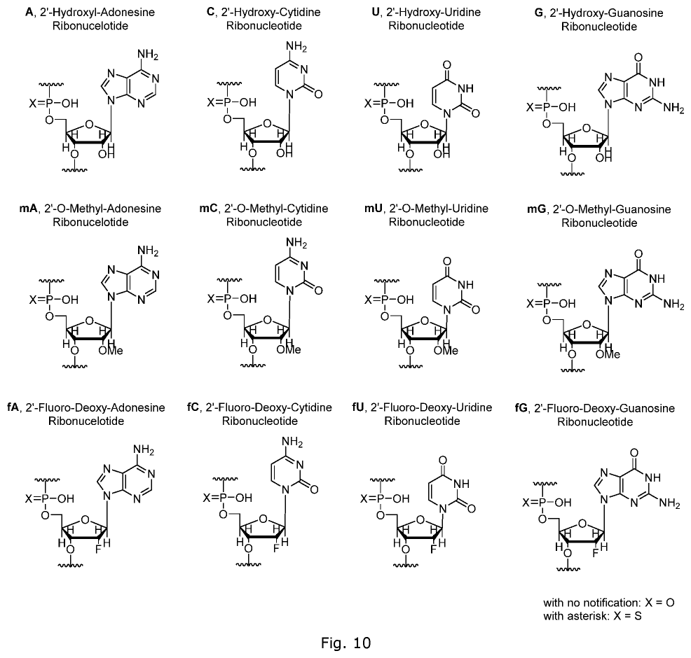

Phosphorylation described herein may occur according to one or more sequences shown in FIG. 4 or FIG. 9. The processes or methods of phosphorylating oligonucleotides provided herein may include each of the processes according to FIG. 4 or FIG. 9. For 5′-terminus phosphorylation, a trityl protected oligonucleotide on a support is activated by treatment with acid to remove the trityl protection group at the 5′-terminus. Acid may be selected from an organic acid or inorganic acid. Organic acids include, but are not limited to, formic acid, acetic acid, chloroacetic acid, dichloroacetic acid, trichloroacetic acid, trifluoroacetic acid, propionic acid, butyric acid, lactic acid, oxalic acid, succinic acid, fumaric acid, malic acid, tartaric acid, maleic acid, sorbic acid, pyruvic acid, citric acid, benzoic acid, or salicylic acid. Inorganic acids include, but not limited to, hydrochloric acid, hydrofluoric acid, nitric acid, sulfuric acid, or phosphoric acid. In some embodiments, the acid includes acetic acid, chloroacetic acid, dichloroacetic acid, trichloroacetic acid, or trifluoroacetic acid. In some embodiments, the acid is dichloroacetic acid. Support-bound oligonucleotide is reacted with a chemical phosphorylating compound herein in the presence of an activator. In some embodiments, the activator includes, but not limited to, acetic acid, 1H-tetrazole, 5-ethylthio-1H-tetrazole (ETT), 5-benzylthio-1H-tetrazole (BTT), or 4,5-dicyanoimidazole (DCI). In some embodiments, the activator is ETT, DCI, or a combination thereof. In some embodiments, the activator is ETT. After a coupling reaction, unreacted oligonucleotide may be capped with capping reagents. Capping reagent A includes, but is not limited to, acetic anhydride, phenoxyacetic anhydride, or acetyl chloride. Capping reagent B includes 2,6-lutidine, pyridine, or 1-methylimidazole. In some embodiments, capping reagent A is a solution of acetic anhydride and 2,6-lutidine in tetrahydrofuran, and capping reagent B is a solution of 1-methylimidazole in tetrahydrofuran. Oligonucleotide containing 5′-phosphite with a trityl tether on a support is oxidized by an oxidation reagent. In some embodiments, the oxidation reagent includes, but is not limited to, iodine, 10-camphorsulfonyl-oxaziridine (CSO), or tert-butyl hydrogen peroxide. In some embodiments, the oxidizing reagent is a solution of iodine, pyridine, water and tetrahydrofuran. 5′-Phosphorylated oligonucleotide with a trityl tether on a support may be subjected to the process of cleavage and deprotection by ammonolysis reaction using ammonia gas with water under high pressure and high temperature. In some embodiments, the ammonolysis is performed under about 50-60 psi and about 50-60° C., but is not limited to the condition mentioned above. 5′-Phosphorylated oligonucleotide with a trityl tether unbound from a support is extracted with a solution of DMSO, acetonitrile and triethylammonium acetate buffer, but not limited to the condition mentioned above. In some embodiments, for the wildtype RNA oligonucleotides or partially modified RNA oligonucleotides, deprotection of the 2′-O-protection group (which may be tert-butyldimethylsilyl (TBDMS)) may proceed under the condition of DMSO, triethylamine and triethylamine trihydrofluoride at 50-60° C. for about 1 to 6 hours, but not limited to the condition mentioned above. The production of the last coupling can also be quantified by detritylation of the oligonucleotide still anchored to the solid support. Deprotection in this case leads directly to the target 5′-phosphorylated RNA fragments.

Also provided herein are methods of purifying a molecule phosphorylated by a compound provided herein. The compounds herein include a trityl moiety. The hydrophobicity of the trityl moiety facilitates purification. The process for synthetic molecule (e.g., an oligonucleotide) purification may be selected from trityl-on or trityl-off purification depending on the presence of a trityl group in the molecule to be purified. Purification of molecules prepared by cyclic synthetic methods referred to herein suffer from, at least, difficulty in separating the desired full-length synthetic molecule from truncated molecules or molecules having a monomer sequence other than that desired. Typically, high-performance liquid chromatography (HPLC) with a reverse phase column is used to facilitate purification of trityl-on synthesized molecules, and anion exchange column chromatography is used to facilitate purification of trityl-off synthesized molecules. However, such chromatographic purification is time consuming, and a significant amount of desired final product may be lost throughout the chromatographic steps resulting in a lower than desired product yield.

Oligonucleotide possessing 5′-phosphate group with a tritylated tether can be separated from truncated impurities by oligonucleotide purification cartridge (OPC) purification or reverse phase HPLC purification. Oligonucleotide possessing 5′-phosphate group without a tritylated tether which is removed by acid can be separated from truncated impurities by anion exchange HPLC purification. OPC purification may be achieved in less than two hours; HPLC purification may take four or more hours.

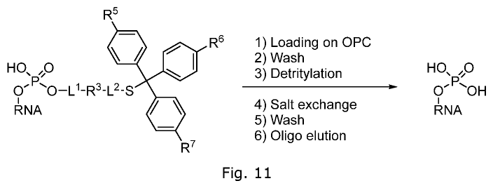

The OPC purification provides a highly purified oligonucleotide containing 5′-phosphate group as depicted in FIG. 5 or FIG. 11. Oligonucleotide containing 5′-phosphate with a trityl tether may be loaded on the OPC with salted solution and the truncated impurities washed with aqueous acetonitrile solution. Successive cleavage of disulfide and removal of ethylene sulfide (thiirane) with gentle reducing reagent solution such as tris(2-carboxyethyl)phosphine (TCEP) generates the 5′-phosphate group in the OPC (FIG. 5), or, alternatively, successive detritylation and removal of ethylene sulfide (thiirane) with gentle acid solution containing scavenger generates the 5′-phosphate group in the OPC (FIG. 11). After washing impurities, a highly purified oligonucleotide containing 5′-phosphate group is eluted with aqueous acetonitrile solution to result in more than 85% purity. Thus, provided herein are methods for phosphorylating an oligonucleotide with high yield and moreover for purifying an oligonucleotide with 5′-phosphate group in high purity utilizing the DMT-ON OPC purification.

The reverse phase HPLC purification provides a highly purified oligonucleotide containing 5′-phosphate group with a tritylated tether. Successive cleavage of disulfide and removal of ethylene sulfide (thiirane) with gentle reducing reagent solution such as tris(2-carboxyethyl)phosphine (TCEP) generates the 5′-phosphorylated oligonucleotides with high purity, or, alternatively, successive detritylation and removal of ethylene sulfide (thiirane) with gentle acid solution generates the 5′-phosphorylated oligonucleotides in more than 95% purity. Thus, provided herein are methods for phosphorylating an oligonucleotide with high yield and for purifying an oligonucleotide with 5′-phosphate group in high purity by reverse phase HPLC purification.

The anion exchange HPLC purification provides a highly purified oligonucleotide containing 5′-phosphate group. Oligonucleotide containing 5′-phosphate with a trityl tether is pre-treated with gentle reducing reagent solution such as tris(2-carboxyethyl)phosphine (TCEP) affording 5′-phosphorylated oligonucleotide, which is loaded on the anion exchange column with salted solution, or, alternatively, oligonucleotide containing 5′-phosphate with a trityl tether is pre-treated with gentle acid solution affording 5′-phosphorylated oligonucleotide, which is loaded on the anion exchange column with salted solution. A highly purified oligonucleotide containing 5′-phosphate group is eluted after anion exchange HPLC purification to result in more than 85% purity or even 95% purity. Thus, provided herein are methods for phosphorylating an oligonucleotide with high yield and moreover for purifying an oligonucleotide with 5′-phosphate group in high purity by anion exchange HPLC purification.

Thus, in some embodiments, the methods of purification provided herein include one or more sequences shown in FIG. 5 or FIG. 11, which may result in a tritylated and phosphorylated molecule or a detritylated and phosphorylated molecule of at least 70%, at least 80%, at least 90%, or at least 95% purity.

In some embodiments, a molecule, such as an oligonucleotide, is 1) prepared by solid phase synthesis, 2) phosphorylated with a compound of Table 1, 3) processed such that the molecule comprises a trityl moiety, 4) loaded onto a column such as an OPC column, 5) the column is washed to remove certain impurities, 6) the molecule loaded on the column is treated with gentle reducing reagent solution such as tris(2-carboxyethyl)phosphine (TCEP) to afford a phosphorylated molecule that is still loaded on the column, 7) the column is washed again to remove certain impurities, and 8) the molecule having a phosphate (—PH₂O₄) (wherein the P is from the compound of Table 1), or a salt thereof, is eluted from the column and isolated as a solution of a substantially purified synthetically prepared molecule having a phosphate (—PH₂O₄), or a salt thereof, or further processed (e.g., a lyophilization step is used) to provide a substantially purified synthetically prepared molecule having a phosphate (—PH₂O₄), or a salt thereof, in a solid form.

The following examples further illustrate aspects of the present disclosure. However, they are in no way a limitation of the teachings or disclosure as described herein.

### Examples

The chemical phosphorylation reagents, e.g., phosphoramidite building blocks, provided herein can be made by any suitable methods. The following describes examples of techniques for producing specific phosphoramidites, for preparing phosphorylated molecules, or for purifying phosphorylated molecules, which techniques may be generalized to produce, prepare, and purify the compounds provided herein. Table 2 includes components of a general method for analytical HPLC, which may be useful in the methods provided herein.

TABLE 2
General method for analytic HPLC method.

Analytical HPLC Condition A: Anion Exchange chromatography
Mobile phase A: 5 mM Tris, 2M Urea, 40% MeCN in water
Mobile phase B: 5 mM Tris, 2M Urea, 0.5M LiClO₄, 40% MeCN in water
Gradient elution of Mobile phase: 100% A to 40% B for 15 minutes
Column: DNAPac PA200
Column temperature: 60° C.
Amount of sample injection: ~0.1 OD
Flow rate: 0.8 mL/min

### Example 1: Synthesis of 2-((bis(4-methoxyphenyl)(phenyl)methyl)thio)ethyl (2-cyanoethyl) diisopropylphosphoramidite (compound 5)

To a solution of DMTr-Cl 18.7 g (55 mmol) in pyridine 106 mL (20 v/w) was added methyl 2-thioglycolate 4.5 mL (50 mmol) at 0° C. for 5 minutes. Reaction mixture was slowly warm up to RT and stirred overnight. After partial evaporation up to 40%, reaction mixture was quenched with ether 150 mL and water 100 mL with stirring. Organic layer was separated, washed with water and brine, dried over anhydrous sodium sulfate and concentrated. Residue was co-evaporated with toluene three times and dried under reduced pressure. Intermediate 21.7 g was obtained as pale yellowish oil, which was used for the next reaction without further purification.

To a solution of above intermediate 21.4 g (50 mmol) in ethanol 82 mL (4 v/w) and THF 82 mL (4 v/w) was added sodium borohydride 2.8 g (75 mmol) at 0° C. Reaction mixture was stirred at 0° C. for 1 hour and refluxed overnight. After quenching with 100 mL of water at 0° C., the resulting solid precipitation was dissolved in ethyl acetate with vigorous stirring. Organic layer was separated, washed with water and brine, dried over anhydrous sodium sulfate, filtered and concentrated. Creamy white oil was column chromatographed on silica gel (pre-treated with 0.5% triethylamine in hexane) with gradient elution of 10˜50% ethyl acetate/hexane containing 1% of triethylamine to yield 12.5 g of alcohol as pale yellowish oil (66%).

Alcohol 12.5 g (32.8 mmol) and diisopropylammonium 1H-tetrazole (DIHT) 6.7 g (39.3 mmol) was dissolved in anhydrous dichloromethane and acetonitrile 125 mL (1:2, 10 v/w) and dried over molecular sieve 3 Å 1.3 g overnight. 2-Cyanoethyl N,N,N′,N′-tetraisopropylphosphorodiamidite 11.5 mL (36.1 mmol) was dissolved in anhydrous dichloromethane and acetonitrile 62 mL (1:2, 5 v/w) and dried over molecular sieve 3 Å 0.62 g overnight. To a solution of alcohol and DIHT was added a solution of 2-cyanoethyl N,N,N′,N′-tetraisopropylphosphorodiamidite at 0° C. for 5 minutes. Reaction mixture was stirred at RT for 3 hours and filtered to remove molecular sieves. Filtrate was quenched with saturated aqueous sodium bicarbonate solution. Organic layer was separated with ethyl acetate. Combined organic layer was washed with water and brine, dried over anhydrous sodium sulfate, filtered and concentrated. Residue was column chromatographed on silica gel (pre-treated with 0.5% triethylamine in hexane) with gradient elution of 5˜20% ethyl acetate/hexane containing 1% of triethylamine to yield 14.4 g of target compound as pale yellowish solid (75%). ¹H NMR (CD3CN, 500 MHz) δ: 7.40-7.38 (m, 2H), 7.31-7.27 (m, 6H), 7.24-7.19 (m, 1H), 6.85-6.83 (m, 4H), 3.76 (s, 6H), 3.75-3.72 (m, 2H), 3.55-3.35 (m, 4H), 2.62-2.59 (m, 2H), 2.39 (t, J=5 Hz, 2H), 1.14 (d, J=10 Hz, 6H), 1.10 (d, J=10 Hz, 6H), ³¹P NMR (CD₃CN, 202 MHz) 147.98 (s), Mass: Calculated for C₃₂H₄₁N₂O₄PS [M+Na]+603.2, Found 603.0.

### Example 2a: Synthesis of 2-cyanoethyl (2-((tris(4-methoxyphenyl)methyl)thio)ethyl) diisopropylphosphoramidite (compound 7)

To a solution of TMTr-Cl 20.3 g (55 mmol) in pyridine 106 mL (20 v/w) was added methyl 2-thioglycolate 4.5 mL (50 mmol) at 0° C. for 5 minutes. Reaction mixture was slowly warm up to RT and stirred overnight. After partial evaporation up to 40%, reaction mixture was quenched with ether 150 mL and water 100 mL with stirring. Organic layer was separated, washed with water and brine, dried over anhydrous sodium sulfate and concentrated. Residue co-evaporated with toluene three times and dried under reduced pressure. Intermediate 21.9 g was obtained as pale yellowish oil, which was used for the next reaction without further purification.

To a solution of intermediate 21.9 g (50 mmol) in ethanol 88 mL (4 v/w) and THF 88 mL (4 v/w) was added sodium borohydride 5.7 g (150 mmol) at 0° C. Reaction mixture was stirred at 0° C. for 1 hour and refluxed overnight. After quenching with 100 mL of water at 0° C., the resulting solid precipitation was dissolved in ethyl acetate with vigorous stirring. Organic layer was separated, washed with water and brine, dried over anhydrous sodium sulfate, filtered and concentrated. Creamy white oil was column chromatographed on silica gel (pre-treated with 0.5% triethylamine in hexane) with gradient elution of 10˜50% ethyl acetate/hexane containing 1% of triethylamine to yield 13.1 g of alcohol as pale yellowish oil (64%).

Alcohol 13.0 g (31.6 mmol) and diisopropylammonium 1H-tetrazole (DIHT) 6.5 g (37.9 mmol) was dissolved in anhydrous dichloromethane and acetonitrile 130 mL (1:2, 10 v/w) and dried over molecular sieve 3 Å 1.3 g overnight. 2-Cyanoethyl N,N,N′,N′-tetraisopopylphosphorodiamiditite 11.1 mL (34.9 mmol) was dissolved in anhydrous dichloromethane and acetonitrile 65 mL (1:2, 5 v/w) and dried over molecular sieve 3 Å 0.65 g overnight. To a solution of alcohol and DIHT was added a solution of 2-cyanoethyl N,N,N′,N′-tetraisopropylphosphorodiamidite at 0° C. for 5 minutes. Reaction mixture was stirred at RT for 3 hours and filtered to remove molecular sieves. Filtrate was quenched with saturated aqueous sodium bicarbonate solution. Organic layer was separated with ethyl acetate. Combined organic layer was washed with water and brine, dried over anhydrous sodium sulfate, filtered and concentrated. Residue was column chromatographed on silica gel (pre-treated with 0.5% triethylamine in hexane) with gradient elution of 5˜20% ethyl acetate/hexane containing 1% of triethylamine to yield 16.5 g of Compound (13) as pale yellowish solid (85%). ¹H NMR (CD₃CN, 500 MHz) δ: 7.29-7.26 (m, 6H), 6.85-6.82 (m, 6H), 3.77 (s, 9H), 3.76-3.71 (m, 2H), 3.54-3.31 (m, 4H), 2.62-2.59 (m, 2H), 2.41-2.38 (m, 2H), 1.14 (d, J=10 Hz, 6H), 1.10 (d, J=10 Hz, 6H), ³¹P NMR (CD₃CN, 202 MHz) 147.96 (s), Mass: Calculated for C₃₃H₄₃N₂O₅PS [M+Na]+633.25, Found 633.1.

### Example 2b: Synthesis of 2-((2-(bis(4-methoxyphenyl)(phenyl)methoxy)ethyl)disulfaneyl)ethyl (2-cyanoethyl) diisopropylphosphoramidite

To a solution of DMTr-Cl 2.42 g (7.1 mmol) in pyridine 29.7 mL was added bis-hydroxyethyl disulfide 1.00 g (6.5 mmol) at 0° C. for 5 minutes. Reaction mixture was slowly warm up to RT and stirred for additional 1 hour. Reaction mixture was quenched with methanol 3 mL at 0° C. and concentrated under the reduced pressure. Resulting residue was purified by column chromatography on silica gel with 0˜10% Methanol in DCM with 01.% triethylamine. 2.57 g of mono DMT protected intermediate was obtained (86.5%).

Mono DMT protected intermediate 2.28 g (5.0 mmol) was dissolved in anhydrous THF 22.8 mL and dried over molecular sieve 3 Å 1 g overnight. To the solution were added N,N′-diisopropylethylamine 4.4 mL (25.1 mmol) and 2-cyanoethyl N,N′-(diisopropyl)phosphoramidochloridite 1.23 mL (5.5 mmol) at 0° C. Reaction mixture was stirred at 0° C. for 1 hour, and then diluted with ethyl acetate 45 mL. The organic layer was washed with saturated aqueous sodium bicarbonate 10 mL, and brine 10 mL. The combined aqueous layer was back-extracted with ethyl acetate 20 mL. The combined organic layer was dried over anhydrous sodium sulfate and concentrated. The resulting residue was quickly purified by short-pad silica gel column chromatography (10% ethyl acetate in hexane with 1% triethylamine) to afford the target compound as pale yellowish solid 2.20 g (69%).

### Example 3: Synthesis of (2-((2-((4-methoxyphenyl)diphenylmethoxy)ethyl)disulfaneyl)ethyl) (2-cyanoethyl) diisopropylphosphoramidite (compound 12)

To a solution of MMTr-Cl 6.67 g (21.6 mmol) in pyridine 89.2 mL was added bis-hydroxyethyl disulfide 2.78 g (18.0 mmol) at 0° C. for 5 minutes. Reaction mixture was slowly warm up to RT and stirred overnight. Reaction mixture was quenched with methanol 10 mL at 0° C. and concentrated under the reduced pressure. Resulting residue was purified by column chromatography on silica gel with 0˜10% Methanol in DCM with 0.1% triethylamine. 6.52 g of mono MMT protected intermediate was obtained (85%).

Mono MMT protected intermediate 2.13 g (5.0 mmol) was dissolved in anhydrous THF 21.3 mL and dried over molecular sieve 3 Å 1 g overnight. To the solution were added N,N′-diisopropylethylamine 4.4 mL (25.1 mmol) and 2-cyanoethyl N,N′-(diisopropyl)phosphoramidochloridite 1.23 mL (5.5 mmol) at 0° C. Reaction mixture was stirred at 0° C. for 1 hour, and then diluted with ethyl acetate 45 mL. The organic layer was washed with saturated aqueous sodium bicarbonate 10 mL, and brine 10 mL. The combined aqueous layer was back-extracted with ethyl acetate 20 mL. The combined organic layer was dried over anhydrous sodium sulfate and concentrated. The resulting residue was quickly purified by short-pad silica gel column chromatography (10% ethyl acetate in hexane with 1% triethylamine) to afford the target compound as pale yellowish solid 2.20 g (69%).

### Example 4: Synthesis of (2-((2-(trityloxy)ethyl)disulfaneyl)ethyl) (2-cyanoethyl) diisopropylphosphoramidite (compound 10)

To a solution of Trityl-Cl 6.02 g (21.6 mmol) in pyridine 81.0 mL was added bis-hydroxyethyl disulfide 2.78 g (18.0 mmol) at 0° C. for 5 minutes. Reaction mixture was slowly warm up to RT and stirred overnight. Reaction mixture was quenched with methanol 10 mL at 0° C. and concentrated under the reduced pressure. Resulting residue was purified by column chromatography on silica gel with 0˜10% Methanol in DCM with 0.1% triethylamine. 6.06 g of mono MMT protected intermediate was obtained (85%).

Mono trityl protected intermediate 1.98 g (5.0 mmol) was dissolved in anhydrous THF 19.8 mL and dried over molecular sieve 3 Å 1 g overnight. To the solution were added N,N′-diisopropylethylamine 4.4 mL (25.1 mmol) and 2-cyanoethyl N,N′-(diisopropyl)phosphoramidochloridite 1.23 mL (5.5 mmol) at 0° C. Reaction mixture was stirred at 0° C. for 1 hour, and then diluted with ethyl acetate 45 mL. The organic layer was washed with saturated aqueous sodium bicarbonate 10 mL, and brine 10 mL. The combined aqueous layer was back-extracted with ethyl acetate 20 mL. The combined organic layer was dried over anhydrous sodium sulfate and concentrated. The resulting residue was quickly purified by short-pad silica gel column chromatography (10% ethyl acetate in hexane with 1% triethylamine) to afford the target compound as pale yellowish solid 1.88 g (55%).

### Example 5: General Procedure for the Preparation of Oligonucleotide Containing 5′-Phosphate Group with a Tritylated Tether Using Solid Phase Synthesis

Oligonucleotide containing 5′-phosphate group with a tritylated tether was synthesized using standard procedures and described briefly as following; solid support functionalized with 5′-O-trityl, or subsequent RNA monomers were activated by treatment with 3% dichloroacetic acid (DCA) in dichloromethane for 2 minutes. Followed by coupling of a chemical phosphorylating reagent provided herein used at an adequate concentration (0.1 M) using a 4-fold molar excess and a 20-fold molar excess of activator 1H-tetrazole (0.25 M) for 15 minutes three times. The support was oxidized with a 0.02 M iodine solution for 1 minute and was capped using methylimidazole and acetic anhydride/2,6-lutidine in tetrahydrofuran for 1 minute. Deprotection and cleavage from support was accomplished with pretreatment of support with 5% diethylamine followed by incubation in a gas chamber containing water and ammonia gas for 5 hours at 60° C. under high pressure (˜55 psi). The oligonucleotide unbound from support were carefully extracted with a 1:2:2 solution of DMSO, acetonitrile and 0.1 M triethylammonium acetate buffer solution (pH 7) and concentrated to afford the free oligonucleotide containing 5′-phosphate group with a tritylated tether. Oligonucleotide containing 5′-phosphate group with a tritylated tether was dissolved in DMSO at 65° C. for 5 minutes and treated with triethylamine and triethylamine trihydrofluoride at 55° C. for 2 hours for 2′-O-tert-butyldimethylsilyl (TBDMS) deprotection, followed by neutralization and evaporation, affording the crude 5′-phosphorylated oligonucleotide with a trityl tether. Alternatively, 2′-fully modified oligonucleotide was transferred to the purification process without further TBDMS deprotection.

### Example 6: General Procedure for the OPC Purification (Process B)

The crude oligonucleotide containing 5′-phosphate group with a trityl tether was purified by oligonucleotide purification cartridge (OPC). A solution of 2.4% triethylamine, 3% N,N-dimethylformamide and 10% sodium chloride in water was transferred to the crude oligonucleotide. Well mixed solution was loaded onto the OPC. A truncated oligonucleotide waste was washed by about 5-15% acetonitrile in 2 M triethylammonium acetate (pH 7) solution while the oligonucleotide containing 5′-phosphate group with a tritylated tether was retained by the cartridge packing material. Tritylated tether was removed by eluting with an about 1-30% aqueous TCEP solution. Finally, the oligonucleotide containing 5′-phosphate group was eluted by 30% acetonitrile in 0.1 M ammonium bicarbonate solution affording the not less than 85% purity of oligonucleotide. This process is summarized in Table 3. In some embodiments, this general procedure is applied to compounds where R³ is SS.

TABLE 3
OPC Purification process (10 μmol scale) (process B).

| Procedure | | Description |
|---|---|---|
| Sample preparation | | Add 0.36M Tris-base, 0.6M TEAA (pH 7) buffer to |
| | | the DMT-On (trityl-on) oligonucleotide (20 mL) |
| Cartridge preparation | | Place the OPC to the manifold with collection tubes |
| | | in the rack |
| | | Turn on the vacuum (3~7 mmHg) |
| | | Condition the cartridge using acetonitrile 5 mL × 2 |
| | | times and 0.1M TEAA (pH 7) 5 mL × 2 times |
| Purification | Loading | Apply the oligo in loading solution to the cartridge |
| procedure | Wash | Wash the cartridge with 5 mL of 10% acetonitrile |
| | | in 2M TEAA (pH 7) 2 times |
| | | Wash the cartridge with 5 mL of 15% acetonitrile |
| | | in 2M TEAA (pH 7) 2 times |
| | Drying | Dry the cartridge under the reduced pressure |
| | Reduction & | Wash the cartridge with 15 mL of 10% TCEP in |
| | Dethioethylation | water (w/v) rapidly under the elevated vacuum |
| | | (8 mmHg) for 5 minutes |
| | | Wash the cartridge with 15 mL of the same |
| | | solution slowly under the gravity flow or low |
| | | vacuum (1~3 mmHg) for 25 minutes |
| | Wash | Wash the cartridge with 15 mL of 5% acetonitrile |
| | | in 1M (NH₄)HCO₃ solution under the gravity flow |
| | | or low vacuum (1~3 mmHg) for 20 minutes |
| | Drying | Dry the cartridge under the reduced pressure |
| | Elution | Place the appropriate receptacle into the manifold |
| | | Elute the purified 5′-phosphorylated oligonucleotide |
| | | using 17% acetonitrile in 1M (NH₄)HCO₃ solution |
| | | under the gravity flow or low vacuum (1~3 mmHg) |
| | | for 20 minutes |

| Evaluation | Determine the yield and purity |
|---|---|
| | Store the purified oligonucleotide lyophilized solid |
| | at −20° C. |

### Example 6a: Another General Procedure for the OPC Purification (Process A)

The crude oligonucleotide containing 5′-phosphate group with a trityl tether was purified by oligonucleotide purification cartridge (OPC). A solution of 2.4% triethylamine, 3% N,N-dimethylformamide and 10% sodium chloride in water was transferred to the crude oligonucleotide. Well mixed solution was loaded onto the OPC. A truncated oligonucleotide waste was washed by about 5-15% acetonitrile in 2 M triethylammonium acetate (pH 7) solution while the oligonucleotide containing 5′-phosphate group with a tritylated tether was retained by the cartridge packing material. Tritylated tether was removed by 15% dichloroacetic acid and 5% dithiothreitol (as scavenger) in dichloromethane (or acetone) and the released trityl tether and ethylene sulfide (thiirane) was washed off by consequentially 5% diisopropylethylamine in dichloromethane (or acetone), 10% lithium perchlorate in acetone, and acetone only. Finally, oligonucleotide containing 5′-phosphate group was eluted by 30% acetonitrile in 0.1 M ammonium bicarbonate solution affording the 85% purity or even more than 95% purity of oligonucleotide. See Table 4, Table 5, and Table 6. In some embodiments, this general procedure is applied to compounds where R⁴ is S.

TABLE 3a
OPC Purification process (1 μmol scale)(process A)

| Procedure | | Description |
|---|---|---|
| Sample preparation | | Add 0.36M Tris-base, 0.6M TEAA (pH 7) buffer to |
| | | the DMT-On (trityl-on) oligonucleotide (2 mL) |
| Cartridge preparation | | Place the OPC to the manifold with collection tubes |
| | | in the rack |
| | | Turn on the vacuum (3~7 mmHg) |
| | | Condition the cartridge using acetonitrile 500 μL × 2 |
| | | times and 0.1M TEAA (pH 7) 500 μL × 2 times |
| Purification | Loading | Apply the oligo in loading solution to the cartridge |
| procedure | Wash | Wash the cartridge with 500 μL of 10% acetonitrile |
| | | in 2M TEAA (pH 7) 2 times |
| | | Wash the cartridge with 500 μL of 15% acetonitrile |
| | | in 2M TEAA (pH 7) 2 times |
| | Drying | Dry the cartridge under the reduced pressure |
| | Detritylation | Wash the cartridge with 1.5 mL of 10% DCA, 8% |
| | | DTT in DCE (w/w/v) rapidly under the elevated |
| | | vacuum (8 mmHg) for 5 minutes |
| | | Wash the cartridge with 1.5 mL of the same |
| | | solution slowly under the gravity flow or low |
| | | vacuum (1~3 mmHg) for 25 minutes |
| | Dethioethylation | Wash the cartridge with 1.5 mL of 5% DIPEA, 4% |
| | (optional) | DTT in DCE (v/w/v) for 5~15 minutes if the scale is |
| | | larger than 1 μmol |
| | Wash | Wash the cartridge with 500 μL of DCE three times |
| | | Wash the cartridge with 500 μL of DCM three times |
| | | Wash the cartridge with 500 μL of acetone three |
| | | times |
| | Drying | Dry the cartridge under the reduced pressure |
| | Elution | Place the appropriate receptacle into the manifold |
| | | Elute the purified 5′-phosphorylated oligonucleotide |
| | | using 17% acetonitrile in 1M (NH₄)HCO₃ solution |
| | | under the gravity flow or low vacuum (1~3 mmHg) |
| | | for 20 minutes |

| Evaluation | Determine the yield and purity |
|---|---|
| | Store the purified oligonucleotide lyophilized solid |
| | at −20° C. |

### Example 6b: Wildtype and Modified RNA Oligonucleotides

Oligonucleotides prepared herein include sequences selected from Table 3c.

TABLE 3c
Wildtype RNA or 2′-OMe
modified RNA oligonucleotides.

| | |
|---|---|
| SEQ ID NO: 1 | 5′- C A C A G U G A U C G |
| | G C A U U-3′ |
| SEQ ID NO: 2 | 5′- A A U G U G U G A A C |
| | G U G U U-3′ |
| SEQ ID NO: 3 | 5′- C A G U G A U C A G C |
| | A U U C U-3′ |
| SEQ ID NO: 4 | 5′- A G A A U G U G A U C |
| | A C U G U-3′ |
| SEQ ID NO: 5 | 5′- A G G C C U G C A G A |
| | U C U U U-3′ |
| SEQ ID NO: 6 | 5′- A A U U G A G C A A C |
| | A G G C C-3′ |
| SEQ ID NO: 7 | 5′- A G U C C A A U U U C |
| | C A G C A-3′ |
| SEQ ID NO: 8 | 5′- U U U U G C U A U U G |
| | G C C U U-3′ |
| SEQ ID NO: 9 | 5′- G C C A A U U U C C A |
| | G C A A A-3′ |
| SEQ ID NO: 10 | 5′- C A C A G U G A U C G |
| | G C A U U-3′ |
| SEQ ID NO: 11 | 5′- G U A U C C U A A U G |
| | G U G U A-3′ |
| SEQ ID NO: 12 | 5′-mG mU mA U C C U A A U |
| | G mG U G U mA-3′ |
| SEQ ID NO: 13 | 5′-mG mU mA mU mC mC mU mA |
| | mA mU mG mG mU mG mU mA-3′ |

### Example 7: 5′-Phosphorylated Wildtype RNA Oligonucleotides were Obtained with High Purity by High-Throughput or Large Scale OPC Purification

5′-[Tr-SS-Phos]-[SEQ ID NO:11]-3′ are prepared using Compound 10 and purified by the OPC purification process B in 10 μmol scale. Results are shown below in Table 4. Similar results are obtained when a phosphorylating reagent provided herein is used that includes R³ as SS.

### Example 8: 5′-Phosphorylated Partially Modified RNA Oligonucleotides were Obtained with High Purity by High-Throughput or Large Scale OPC Purification

5′-[Tr-SS-Phos]-[SEQ ID NO:12]-3′ are prepared using Compound 10 and purified by the OPC purification process B in 10 μmol scale. Results are shown below in Table 4. Similar results are obtained when a phosphorylating reagent provided herein is used that includes R³ as SS.

### Example 9: 5′-Phosphorylated Fully Modified RNA Oligonucleotides were Obtained with High Purity by High-Throughput or Large Scale OPC Purification

5′-[Tr-SS-Phos]-[SEQ ID NO:13]-3′ are prepared using Compound 10 and purified by the OPC purification process B in 10 μmol scale. Results are shown below in Table 4. Similar results are obtained when a phosphorylating reagent provided herein is used that includes R³ as SS.

TABLE 4
OPC purification results (AEX HPLC) for 3 strands
of 5′-phosphorylated RNA oligonucleotides with
trityl disulfide tether in 10 μmol scale

| Sample ID | SEQ ID NO | Description | Purity |
|---|---|---|---|
| A (Example 7) | 11 | Wildtype, Crude | 49.2% |
| B (Example 7) | 11 | Wildtype, OPC Purified | 88.2% |
| C (Example 8) | 12 | Partially modified, Crude | 51.4% |
| D (Example 8) | 12 | Partially modified, Purified | 91.4% |
| E (Example 9) | 13 | Fully modified, Crude | 58.1% |
| F (Example 9) | 13 | Fully modified, Purified | 93.7% |

### Example 9a: High-Throughput OPC Purification Process A

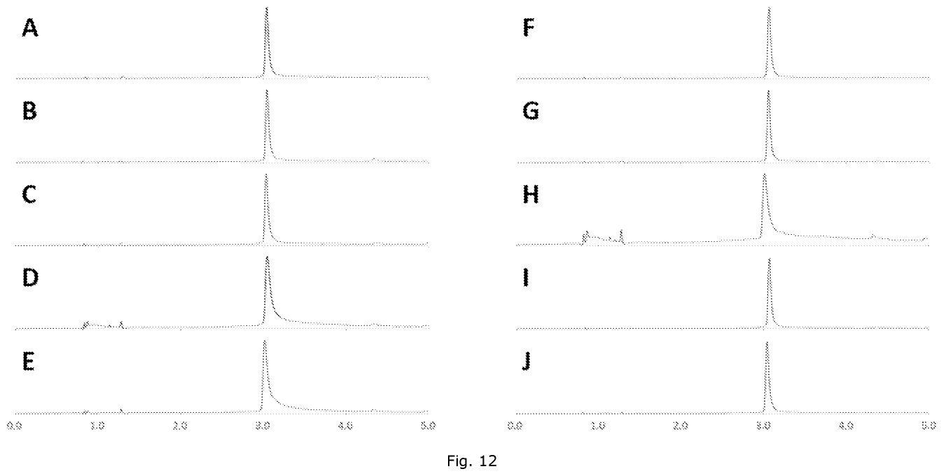

10 wildtype strands of 5′-phosphorylated RNA oligonucleotides with DMTr thioethyl tether were prepared at 100 nmol scale using Compound 5 as the phosphorylation reagent. High-throughput OPC purification was performed using purification process A. Results are shown below in Table 5, and in FIG. 12. Similar results are obtained when a phosphorylating reagent provided herein is used that includes R⁴ as S.

TABLE 5
OPC purification results (LC/MS) for 10 wildtype
strands of 5′-phosphorylated RNA oligonucleotides
with DMTr thioethyl tether in 100 nmol scale.

| Sample ID | SEQ ID NO | Purity |
|---|---|---|
| A | 1 | 99.1% |
| B | 2 | 98.1% |
| C | 3 | 97.9% |
| D | 4 | 98.0% |
| E | 5 | 99.0% |
| F | 6 | 97.6% |
| G | 7 | 99.5% |
| H | 8 | 97.0% |
| I | 9 | 99.4% |
| J | 10 | 99.6% |

### Example 9b: 5′-Phosphorylated RNA Oligonucleotides with High Purity by Large Scale OPC Purification Process a

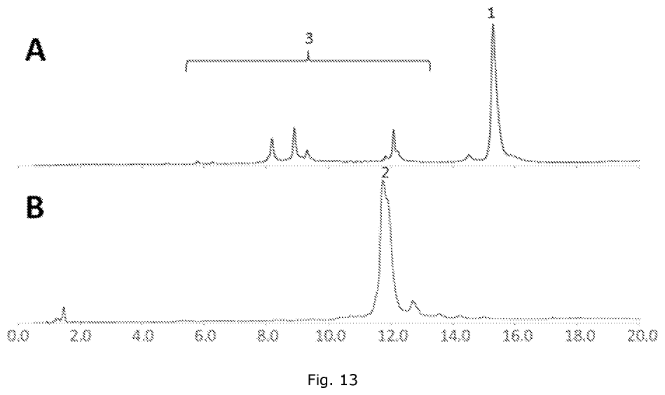

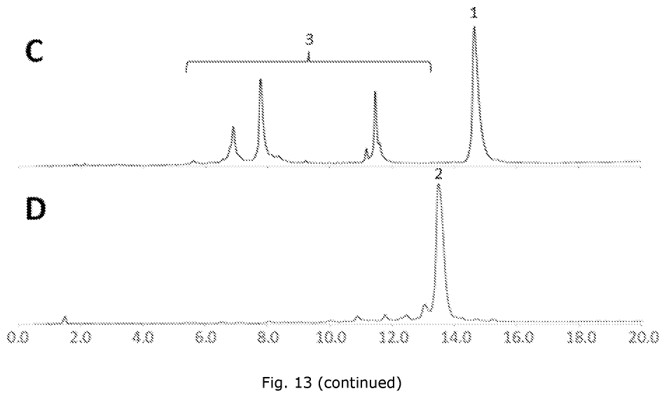

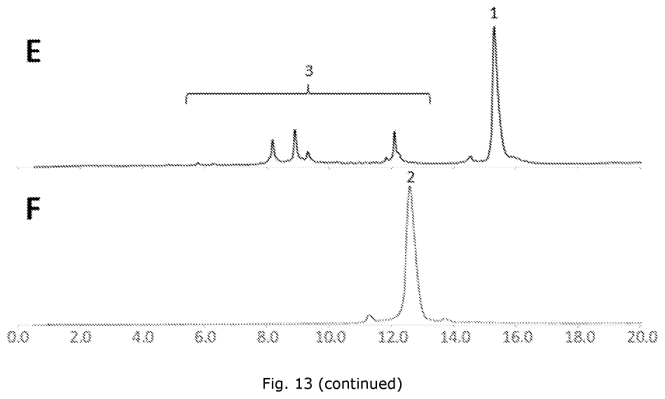

5′-[DMTr-TE-Phos]-[SEQ ID NO: 11]-3′, 5′-[DMTr-TE-Phos]-[SEQ ID NO: 12]-3′, and 5′-[DMTr-TE-Phos]-[SEQ ID NO:13]-3′ are prepared using compound 5 as the phosphorylation reagent, and purified by the OPC purification process A in 10 μmol scale. Results are shown below in Table 6, and in FIG. 13. Similar results are obtained when a phosphorylating reagent provided herein is used that includes R⁴ as S.

TABLE 6
OPC purification results (AEX HPLC) for 10 wildtype
strands of 5′-phosphorylated RNA oligonucleotides
with DMTr thioethyl tether in 10 μmol scale.

| Sample ID | SEQ ID NO | Description | Purity |
|---|---|---|---|
| A | 11 | Wildtype, Crude | 61.5% |
| B | 11 | Wildtype, OPC Purified | 94.9% |
| C | 12 | Partially modified, Crude | 48.6% |
| D | 12 | Partially modified, Purified | 95.2% |
| E | 13 | Fully modified, Crude | 46.1% |
| F | 13 | Fully modified, Purified | 97.5% |

### Example 9c: High-Throughput OPC Purification Process B

10 wildtype strands of 5′-phosphorylated RNA oligonucleotides with trityl tether were prepared at 100 nmol scale using Compound 10 as the phosphorylation reagent. High-throughput OPC purification was performed using purification process B. Results are shown below in Table 7. Similar results are obtained when a phosphorylating reagent provided herein is used that includes R³ as SS.

TABLE 7
OPC purification results (LC/MS) for 10 wildtype
strands of 5′-phosphorylated RNA oligonucleotides
with Tr disulfide tether in 100 nmol scale.

| SEQ ID NO | Purity |
|---|---|
| 1 | 93.0% |
| 2 | 89.3% |
| 3 | 86.6% |
| 4 | 86.0% |
| 5 | 88.2% |
| 6 | 93.4% |
| 7 | 94.6% |
| 8 | 89.3% |
| 9 | 93.8% |
| 10 | 86.7% |

### Example 10: OPC Purification Using Solid CPR II for 5′-Phosphorylated Wildtype RNA Oligonucleotide

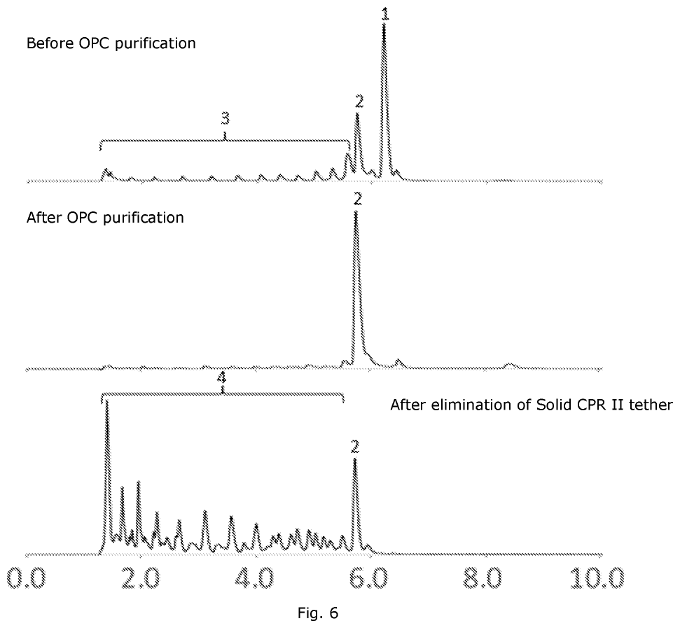

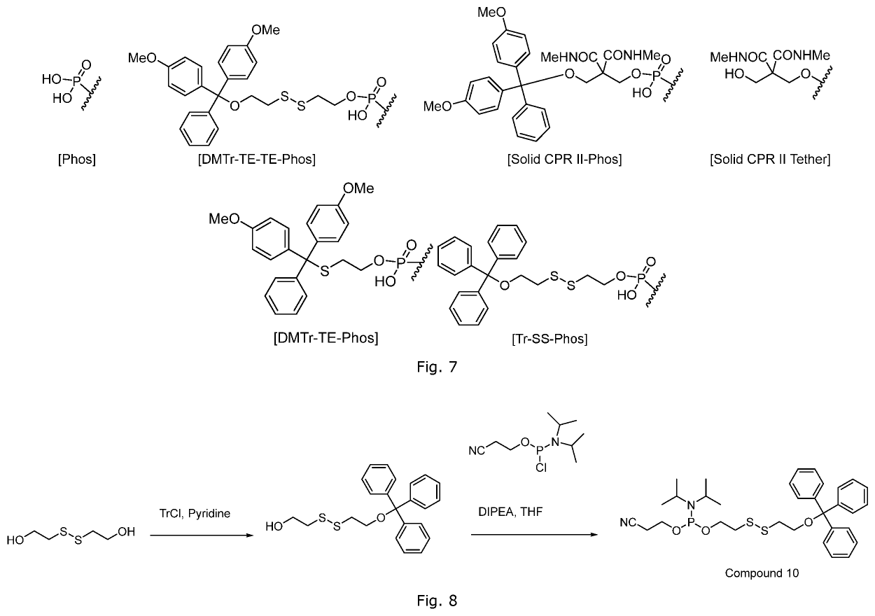

5′-[Solid CPR II-Phos]-[SEQ ID NO:11]-3′ was prepared. The wildtype RNA oligonucleotide containing 5′-phosphate group with a tritylated tether according Solid CPR II was purified by the OPC as the same method described in EXAMPLE 6. Purified oligonucleotide was further incubated under one of conditions selected from ammonium hydroxide for 2 hours at 55° C., AMA for 10 minutes at 65° C., or 0.1 M NaOH at room temperature to eliminate the tritylated tether according to the recommended guideline from Glen Research. As shown in FIG. 6, those elimination condition of Solid CPR II tether lead to decomposition of oligonucleotide containing 5′-phosphate to show only a trace amount of target compound. The steps analyzed for OPC purification using Solid CPR II were: A) before OPC purification; B) after OPC purification; and C) after elimination of Solid CPR II tether under 0.1 N NaOH at RT for 2 hours. FIG. 6 peak #index: 1) 5′-[Solid CPR II-Phos]-[SEQ ID NO:11]; 2) 5′-[Phos]-[SEQ ID NO:11]; 3) failure sequences from oligonucleotide synthesis; and 4) decomposed sequences during the elimination of Solid CPR II Tether). Similar degradation results were observed when wild-type RNA oligonucleotide containing 5′-phosphate group with a tritylated tether by Solid CPR II was purified by the OPC as the same method described in EXAMPLE 6a.

### Example 11: RP HPLC Purification Using Compound 10 for 5′-Phosphorylated Wildtype RNA Oligonucleotide

5′-[Tr-SS-Phos]-[SEQ ID NO:11]-3′ was prepared using Compound 10. The oligonucleotide containing 5′-phosphate group with a tritylated disulfide tether was purified by reverse phase (RP) HPLC. Crude 5′-phosphorylated oligonucleotide with a tritylated tether was dissolved in aqueous acetonitrile solution and pre-filtered over 0.2 μm filtration kit. The filtrate was injected for purification by reverse phase HPLC. All portions containing the target oligonucleotide were analyzed by analytical HPLC and portions containing over 90% purity of target compound were collected and concentrated affording 5′-phosphorylated oligonucleotide with a trityl thioethyl tether. Reduction and dethioethylation was accomplished with 10% aqueous TCEP (w/v) solution over 15 minutes. Resulting 5′-phosphorylated oligonucleotide was desalted to afford the 5′-phosphorylated oligonucleotide in >90% purity. General RP column HPLC purification condition includes the protocols shown in Table 8.

TABLE 8
General HPLC condition: reverse phase
(RP) HPLC purification conditions.

Mobile phase A: 10% acetonitrile in 100 mM TEAA buffer (pH 7.0)
Mobile phase B: 90% acetonitrile in 100 mM TEAA buffer (pH 7.0)
Gradient elution of Mobile phase: 100% A to 100% B for 30 minutes
Column: C18 RP column
Column temperature: 40° C.
Amount of sample injection: 30~300 OD
Flow rate: 1.0 mL/min
UV detector: 260 nm

### Example 12: AEX HPLC Purification Using Compound 10 for 5′-Phosphorylated Wildtype RNA Oligonucleotide

5′-[Tr-SS-Phos]-[SEQ ID NO:11]-3′ was prepared. The oligonucleotide containing 5′-phosphate group without a tritylated disulfide tether was purified by HPLC using anion exchange (AEX) column. Crude 5′-phosphorylated oligonucleotide with a trityl disulfide tether was treated with 10% aqueous TCEP (w/v) solution over 15 minutes to afford the crude 5′-phosphorylated oligonucleotide. After partial concentration and pre-filtration over 0.2 μm filtration kit, the filtrate was injected for purification by HPLC using AEX column. All portions containing the target oligonucleotide were analyzed by analytical HPLC and portions containing over 90% purity of target compound were collected and concentrated affording 5′-phosphorylated oligonucleotide in >90% purity. General AEX column HPLC purification condition includes the protocols shown in Table 9.

TABLE 9
General HPLC Condition: anion exchange
(AEX) column chromatography.

Mobile phase A: 20 mM Na₂HPO₄, 15% MeCN in water
Mobile phase B: 20 mM Na₂HPO₄, 1M NaBr, 15% MeCN in water
Gradient elution of Mobile phase: 0% B to 40% B for 30 minutes
Column: Anion exchange column
Column temperature: 60° C.
Amount of sample injection: 30~300 OD
Flow rate: 0.8 mL/min
UV detector: 260 nm

TABLE 10
Synthesis and Purification Summary for 5′-[Phos]-RNA Oligonucleotides

| Example | | | | Crude | | Mass | | Final |
|---|---|---|---|---|---|---|---|---|
| No. | Sequence Structure (5′ → 3′) | Scale | | Purity | Method | Calcd | Measd | Purity |
| Example 9a | [DMTr-TE-Phos]-[SEQ ID NO: 1] | 100 | nmol | N/A | OPC | 5161.0 | 5161.1 | 99.1% |
| | [DMTr-TE-Phos]-[SEQ ID NO: 2] | 100 | nmol | N/A | OPC | 5203.0 | 5203.1 | 98.1% |
| | [DMTr-TE-Phos]-[SEQ ID NO: 3] | 100 | nmol | N/A | OPC | 5121.9 | 5121.9 | 97.9% |
| | [DMTr-TE-Phos]-[SEQ ID NO: 4] | 100 | nmol | N/A | OPC | 5186.0 | 5185.9 | 98.0% |
| | [DMTr-TE-Phos]-[SEQ ID NO: 5] | 100 | nmol | N/A | OPC | 5137.9 | 5138.0 | 99.0% |
| | [DMTr-TE-Phos]-[SEQ ID NO: 6] | 100 | nmol | N/A | OPC | 5207.1 | 5207.2 | 97.6% |
| | [DMTr-TE-Phos]-[SEQ ID NO: 7] | 100 | nmol | N/A | OPC | 5105.0 | 5105.1 | 99.5% |
| | [DMTr-TE-Phos]-[SEQ ID NO: 8] | 100 | nmol | N/A | OPC | 5053.8 | 5053.8 | 97.0% |
| | [DMTr-TE-Phos]-[SEQ ID NO: 9] | 100 | nmol | N/A | OPC | 5128.0 | 5128.1 | 99.4% |
| | [DMTr-TE-Phos]-[SEQ ID NO: 10] | 100 | nmol | N/A | OPC | 5202.0 | 5202.2 | 99.6% |
| Example 9b | [DMTr-TE-Phos]-[SEQ ID NO: 11] | 10 | μmol | 61.5% | OPC | 5163.1 | 5163.2 | 94.9% |
| | [DMTr-TE-Phos]-[SEQ ID NO: 12] | 10 | μmol | 48.6% | OPC | 5387.5 | 5387.5 | 95.2% |
| | [DMTr-TE-Phos]-[SEQ ID NO: 13] | 10 | μmol | 46.1% | OPC | 5233.2 | 5233.2 | 97.5% |
| Example 9c | [Tr-SS-Phos]-[SEQ ID NO: 1] | 100 | nmol | N/A | OPC | 5161.0 | 5161.1 | 93.0% |
| | [Tr-SS-Phos]-[SEQ ID NO: 2] | 100 | nmol | N/A | OPC | 5203.0 | 5203.1 | 89.3% |
| | [Tr-SS-Phos]-[SEQ ID NO: 3] | 100 | nmol | N/A | OPC | 5121.9 | 5121.9 | 86.6% |
| | [Tr-SS-Phos]-[SEQ ID NO: 4] | 100 | nmol | N/A | OPC | 5186.0 | 5185.9 | 86.0% |
| | [Tr-SS-Phos]-[SEQ ID NO: 5] | 100 | nmol | N/A | OPC | 5137.9 | 5138.0 | 88.2% |
| | [Tr-SS-Phos]-[SEQ ID NO: 6] | 100 | nmol | N/A | OPC | 5207.1 | 5207.2 | 93.4% |
| | [Tr-SS-Phos]-[SEQ ID NO: 7] | 100 | nmol | N/A | OPC | 5105.0 | 5105.1 | 94.6% |
| | [Tr-SS-Phos]-[SEQ ID NO: 8] | 100 | nmol | N/A | OPC | 5053.8 | 5053.8 | 89.3% |
| | [Tr-SS-Phos]-[SEQ ID NO: 9] | 100 | nmol | N/A | OPC | 5128.0 | 5128.1 | 93.8% |
| | [Tr-SS-Phos]-[SEQ ID NO: 10] | 100 | nmol | N/A | OPC | 5202.0 | 5202.2 | 86.7% |
| Example 7 | [Tr-SS-Phos]-[SEQ ID NO: 11] | 10 | μmol | 49.2% | OPC | 5163.1 | 5163.0 | 88.2% |
| Example 8 | [Tr-SS-Phos]-[SEQ ID NO: 12] | 10 | μmol | 51.4% | OPC | 5387.5 | 5387.5 | 91.4% |
| Example 9 | [Tr-SS-Phos]-[SEQ ID NO: 13] | 10 | μmol | 58.1% | OPC | 5233.2 | 5233.2 | 93.7% |
| Example 10 | [Solid CPR II-Phos]-[SEQ ID NO: 11] | 10 | μmol | 55.0% | OPC | 5163.1 | 5163.2 | Decomposed |
| Example 13 | [DMTr-TE-Phos]-[SEQ ID NO: 11] | 10 | μmol | 61.5% | RP | 5163.1 | 5163.1 | 94.6% |
| Example 14 | [DMTr-TE-Phos]-[SEQ ID NO: 11] | 10 | μmol | 61.5% | AEX | 5163.1 | 5163.1 | 92.1% |
| Example 11 | [Tr-SS-Phos]-[SEQ ID NO: 11] | 10 | μmol | 49.2% | RP | 5163.1 | 5163.2 | 89.9% |
| Example 12 | [Tr-SS-Phos]-[SEQ ID NO: 11] | 10 | μmol | 49.2% | AEX | 5163.1 | 5163.1 | 91.8% |

### Example 13: Comparative Example—Reverse Phase HPLC Purification

5′-[DMTr-TE-Phos]-[SEQ ID NO:11]-3′ was prepared using Compound 5 and purified by reverse phase (RP) HPLC. Crude 5′-phosphorylated oligonucleotide with a trityl thioethyl tether (Compound 5) was dissolved in aqueous acetonitrile solution and pre-filtered over 0.2 μm filtration kit. The filtrate was injected for purification by reverse phase HPLC. All portions containing the target oligonucleotide were analyzed by analytical HPLC and portions containing over 90% purity of target compound were collected and concentrated affording 5′-phosphorylated oligonucleotide with a trityl thioethyl tether. Detritylation and dethioethylation was accomplished with 10% DCA, 8% DTT in DCE over 15 minutes. Resulting 5′-phosphorylated oligonucleotide was desalted to afford the 5′-phosphorylated oligonucleotide in >90% purity. General RP HPLC purification method is as described in Table 11.

TABLE 11
General method for reverse phase (RP) HPLC purification.

Mobile phase A: 10% acetonitrile in 100 mM TEAA buffer (pH 7.0)
Mobile phase B: 90% acetonitrile in 100 mM TEAA buffer (pH 7.0)
Gradient elution of Mobile phase 100% A to 100% B for 30 minutes
Column: C18 RP column
Column temperature: 40° C.
Amount of sample injection: 30~300 OD
Flow rate: 1.0 mL/min
UV detector: 260 nm

### Example 14: Comparative Example—Anion Exchange HPLC Purification

5′-[DMTr-TE-Phos]-[SEQ ID NO:11]-3′ was prepared using Compound 5 and purified by HPLC using anion exchange (AEX) column. Crude 5′-phosphorylated oligonucleotide with a trityl thioethyl tether was treated with 10% DCA, 8% DTT in DCE over 15 minutes to afford the crude 5′-phosphorylated oligonucleotide. After partial concentration and pre-filtration over 0.2 μm filtration kit, the filtrate was injected for purification by HPLC using AEX column. All portions containing the target oligonucleotide were analyzed by analytical HPLC and portions containing over 90% purity of target compound were collected and concentrated affording 5′-phosphorylated oligonucleotide in >90% purity. General AEX HPLC purification method is as described in Table 12.

TABLE 12
General method for anion exchange (AEX) HPLC purification.

Mobile phase A: 20 mM Na₂HPO₄, 15% MeCN in water
Mobile phase B: 20 mM Na₂HPO₄, 1M NaBr, 15% MeCN in water
Gradient elution of Mobile phase: 0% B to 40% B for 30 minutes
Column: Anion exchange column
Column temperature: 60° C.
Amount of sample injection: 30~300 OD
Flow rate: 0.8 mL/min
UV detector: 260 nm

1. A compound, having the formula: 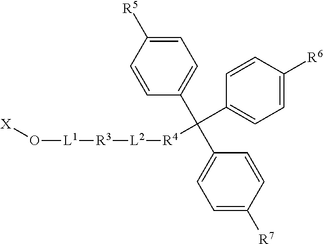
or a salt thereof,
wherein,
X is H, 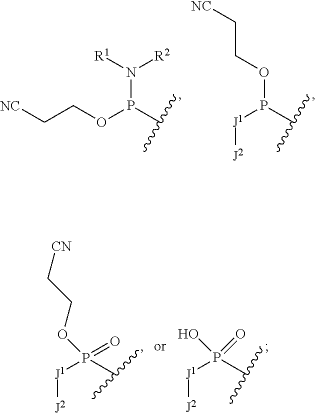
J¹ is O, NH, or S;
J² is a macromolecule;
R¹ is C₁₋₆ alkyl;
R² is C₁₋₆ alkyl;
L¹ is C₁₋₆ alkylene, C₁₋₆ alkenylene, or C₁₋₆ alkynylene;
R³ is a bond, O, N(H), N(CH₃), S, C₂₋₆ alkylene, C₂₋₆ alkenylene, C₂₋₆ alkynylene, SS, O—N═C, C═N—O, C(O)N(H), N(H)C(O), OC(O)O, OC(O)N(H), N(H)C(O)O, N(H)C(O)N(H), OC(O)N(C₁₋₆ alkyl), N(C₁₋₆ alkyl)C(O)O, N(C₁₋₆ alkyl)C(O)N(C₁₋₆ alkyl), C(H)═N, or N═C(H);
L² is C₁₋₆ alkylene, C₁₋₆ alkenylene, or C₁₋₆ alkynylene;
R⁴ is O or S;
R⁵ is H or an electron donating moiety;
R⁶ is H or an electron donating moiety; and
R⁷ is H or an electron donating moiety;
wherein the compound is not 2-((2-(bis(4-methoxyphenyl)(phenyl)methoxy)ethyl)disulfaneyl)ethyl (2-cyanoethyl) diisopropylphosphoramidite or 2-((2-(bis(4-methoxyphenyl)(phenyl)methoxy)ethyl)disulfaneyl)ethan-1-ol.

2. The compound of claim 1, wherein X is H.

3. The compound of claim 1, wherein X is

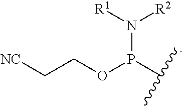

4. The compound of claim 1, wherein X is

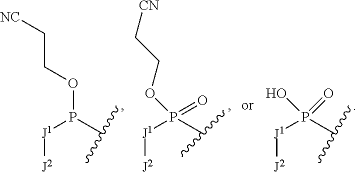

5. The compound of one of claims 1-4, wherein L¹ and L² are different when R³ is SS, R⁴ is O, and L¹ and L² are independently C₁₋₆ alkylene.

6. The compound of one of claims 1-5, wherein R⁴ is O.

7. The compound of one of claims 1-5, wherein R⁴ is S.

8. The compound of one of claims 1-7, wherein R³ is a bond, C₂₋₆ alkylene, C₂₋₆ alkenylene, or C₂₋₆ alkynylene.

9. The compound of one of claims 1-7, wherein R³ is O—N═C, C═N—O, C(O)N(H), N(H)C(O), OC(O)O, OC(O)N(H), N(H)C(O)O, N(H)C(O)N(H), OC(O)N(C₁₋₆ alkyl), N(C₁₋₆ alkyl)C(O)O, N(C₁₋₆ alkyl)C(O)N(C₁₋₆ alkyl), C(H)═N, or N═C(H).

10. The compound of one of claims 1-7, wherein R³ is O, N(H), N(CH₃), S, or SS.

11. The compound of one of claims 1-10, wherein R⁵ is H, R⁶ is OCH₃, and R⁷ is OCH₃.

12. The compound of one of claims 1-10, wherein R⁵ is H, R⁶ is H, and R⁷ is OCH₃.

13. The compound of one of claims 1-10, wherein R⁵ is H, R⁶ is H, and R⁷ is H.

14. The compound of one of claims 1-10, wherein R⁵ is OCH₃, R⁶ is OCH₃, and R⁷ is OCH₃.

15. The compound of claim 1, which is

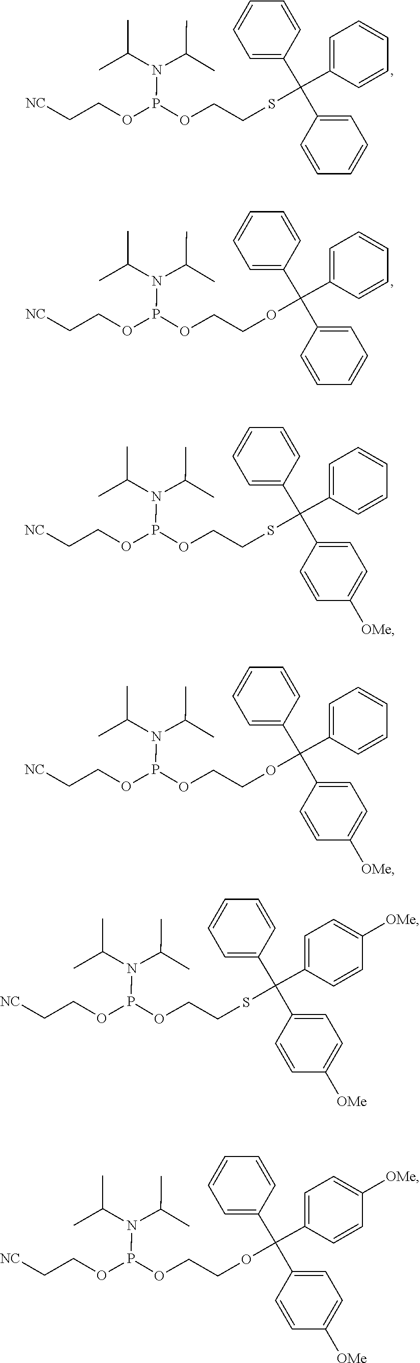 [SMILES: CC(C)N(C(C)C)P(OCCC#N)OCCSC(c1ccccc1)(c1ccccc1)c1ccccc1 ; CC(C)N(C(C)C)P(OCCC#N)OCCOC(c1ccccc1)(c1ccccc1)c1ccccc1 ; COc1ccc(C(SCCOP(OCCC#N)N(C(C)C)C(C)C)(c2ccccc2)c2ccccc2)cc1 ; COc1ccc(C(OCCOP(OCCC#N)N(C(C)C)C(C)C)(c2ccccc2)c2ccccc2)cc1 ; COc1ccc(C(SCCOP(OCCC#N)N(C(C)C)C(C)C)(c2ccccc2)c2ccc(OC)cc2)cc1 ; COc1ccc(C(OCCOP(OCCC#N)N(C(C)C)C(C)C)(c2ccccc2)c2ccc(OC)cc2)cc1]

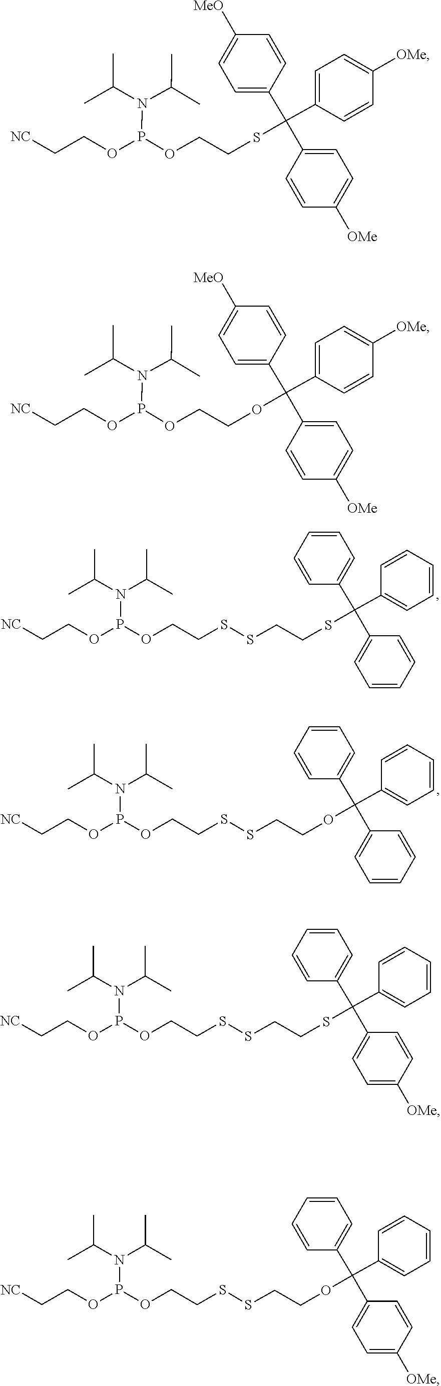

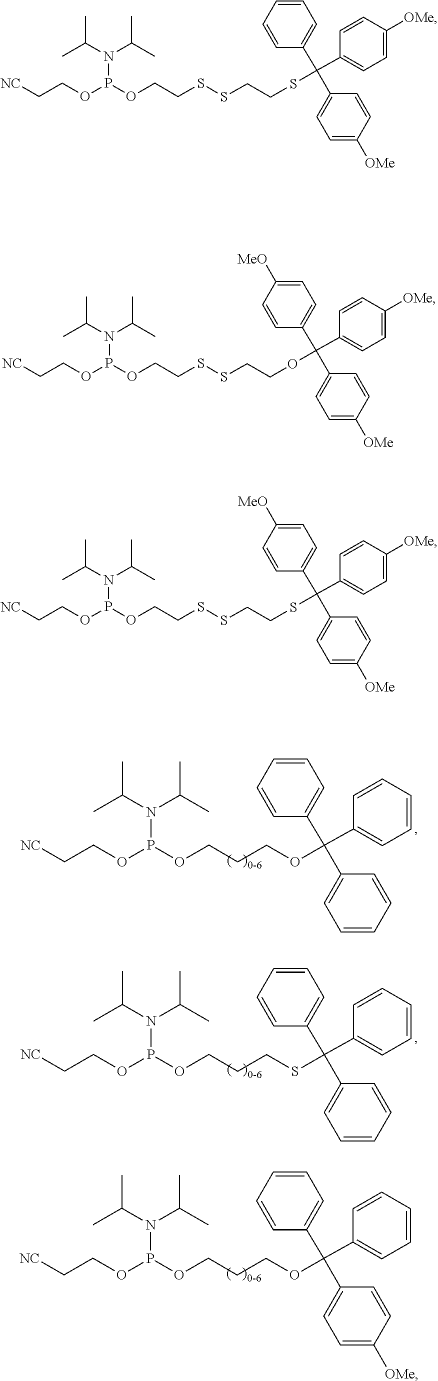

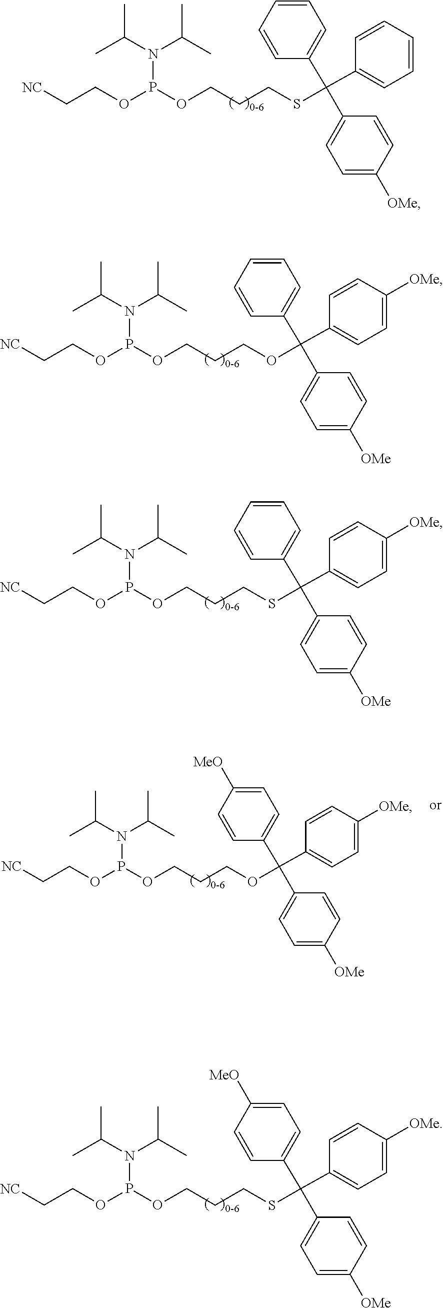

16. A method of preparing a phosphorylated molecule, comprising a coupling step comprising contacting a molecule with the compound of claim 1, 3, or 5-15, which includes a phosphoramidite, optionally in the presence of one or more activators, such that the phosphorous atom of the compound forms a covalent bond with the molecule to form the phosphorylated molecule.

17. The method of claim 16, further comprising a capping step that includes subjecting the phosphorylated molecule to conditions including one or more capping reagents.

18. The method of claim 16 or claim 17, further comprising an oxidation step that includes contacting the phosphorylated molecule with one or more oxidizing reagents.

19. The method of one of claims 16-18, further comprising a deblock step that includes contacting the phosphorylated molecule with one or more acid reagents.

20. The method of one of claims 16-19, further comprising a wash step, independently, after each of the coupling step, the capping step, the oxidation step, or the deblock step, comprising subjecting the phosphorylated molecule to conditions including one or more organic solvents.

21. The method of one of claims 16-20, wherein the molecule is disposed within a container.

22. The method of one of claims 16-21, wherein the molecule comprises a solid support.

23. The method of one of claims 16-22, wherein the molecule comprises an oligomer.

24. The method of one of claims 16-23, wherein the molecule comprises a nucleic acid, a nucleic acid analog, a peptide, or an oligosaccharide.

25. A phosphorylated molecule, prepared by the method of one of claims 16-24.

26. A method of purifying the phosphorylated molecule of claim 25, comprising:
an adsorption step including adsorbing the phosphorylated molecule, which includes a trityl moiety, to an auxiliary solid support.

27. The method of claim 26, further comprising one or more, independently, of:
a washing step that includes subjecting the adsorbed phosphorylated molecule to conditions including one or more organic solvents;
a deblock step that includes contacting the phosphorylated molecule with one or more of a deblocking reagent suitable to remove a moiety from the phosphorylated molecule comprising a trityl moiety; and
an elution step including that includes subjecting the adsorbed phosphorylated molecule to conditions including one or more solvents, wherein the one or more solvents includes an aqueous buffer.

28. The method of claim 27, wherein the deblocking reagent includes one or more of an acid reagent, a tris(2-carboxy C₁₋₆alkyl)phosphine, or dithiothreitol.

29. The method of one of claims 26-28, wherein the phosphorylated molecule was prepared on a scale of about 100 nmol or more.

30. The method of one of claims 26-28, wherein the phosphorylated molecule was prepared on a scale of about 100 nmol, about 1 μmol, about 10 μmol, about 20 μmol, about 0.1 mmol, or greater than 0.15 mmol.

31. A purified phosphorylated molecule, prepared by the method of one of claims 26-30.

32. A composition, comprising the purified phosphorylated molecule of claim 31.

33. The composition of claim 32, which is a pharmaceutical composition comprising at least one pharmaceutical acceptable carrier.

34. The composition of claim 32 or claim 33, wherein the phosphorylated molecule is present at a concentration of about 1 fM or more, and is at least about 70% pure.
